(19) United States
(12) Patent Application Publication
(10) Pub. No.: US 2025/0051413 A1
(43) Pub. Date: Feb. 13, 2025

(54) CHIMERIC MOLECULES COMPRISING IL-12 AGONIST POLYPEPTIDE

(71) Applicant: AskGene Pharma Inc., Camarillo, CA (US)

(72) Inventors: Yuefeng Lu, Newbury Park, CA (US); Chunxiao Yu, Santa Barbara, CA (US); Liqin Liu, Camarillo, CA (US)

(73) Assignee: AskGene Pharma Inc., Camarillo, CA (US)

(21) Appl. No.: 18/272,095

(22) PCT Filed: Jan. 12, 2022
(86) PCT No.: PCT/US2022/012202

§ 371 Date: Jul. 12, 2023

Related U.S. Application Data
(60) Provisional application No. 63/136,640, filed on Jan. 12, 2021.
(60) Provisional application No. 63/137,681, filed on Jan. 14, 2021.
(60) Provisional application No. 63/176,285, filed on Apr. 17, 2021.
(60) Provisional application No. 63/233,222, filed on Aug. 14, 2021.

(51) Int. Cl. C07K 14/54; A61K 38/00; C07K 16/24; C07K 16/28

(52) U.S. Cl. CPC C07K 14/5434; C07K 16/244; C07K 16/2818; A61K 38/00; C07K 2317/569; C07K 2319/50

(57) ABSTRACT

The present application relates to novel chimeric molecules or prodrugs of IL-12. Specifically, disclosed provides a chimeric molecule, which comprises an IL-12 p40 subunit (p40), an IL-12 p35 subunit (p35), and which may further comprises a masking moiety (MM), and/or a carrier (C). Further included in the present application are methods of making and using the novel chimeric molecules or prodrugs.

### CROSS REFERENCE TO RELATED APPLICATION

The present application claims priority to U.S. Provisional Application No. 63/136,640 filed on Jan. 12, 2021, U.S. Provisional Application No. 63/137,681 filed on Jan. 14, 2021, U.S. Provisional Application No. 63/176,285 filed on Apr. 17, 2021, U.S. Provisional Application No. 63/233,222 filed on Aug. 14, 2021, the contents of which are incorporated herein by reference in their entirety.

### REFERENCE TO SEQUENCE LISTING

The entire content of the following electronic submission of the sequence listing via the USPTO EFS-WEB server, as authorized and set forth in MPEP § 1730 II.B.2(a)(C), is hereby expressly incorporated by reference in its entirety for all purposes. The sequence listing is identified on the electronically filed text file as follows: File Name: AG3-026WO_ST25; Date of Creation: Jan. 11, 2022; Size (bytes): 639 KB.

### FIELD OF THE INVENTION

The present application relates to novel chimeric molecules or prodrugs of IL-12. Further included in the present application are methods of making and using the novel chimeric molecules or prodrugs.

### BACKGROUND

IL-12 comprises two subunits, P40 and P35, and plays important roles in immunology (Watford W T et al, Cytokine Growth Factor Rev. 2003 October; 14(5):361-8). It stimulates interferon gamma release and stimulates T cells and NK cells (Del Vecchio M, Clin Cancer Res. 2007 Aug. 15; 13(16):4677-85). IL-12 receptor consists of two subunits, IL-12 receptor beta 1 (IL12Rβ1) and beta 2 (IL12Rβ2). Deficiency of IL12Rβ1 impaired IL-12 signal pathway and may have led to bacterial infection and in one case Sjögren's Syndrome (Sogkas et al., Front Immunol. 2017; 8: 885).

IL-12 has also been explored as potential drug to treat cancer (Colombo M P and Trinchieri G. 2002, Cytokine Growth Factor Rev 13(2):155-168; Del Vecchio M, Clin Cancer Res. 2007 Aug. 15; 13(16):4677-85; Lasek et al, Cancer Immunol Immunother. 2014; 63(5): 419-435).

There is a need to develop IL-12-based-based cancer therapeutics which are more tumor site-selective, has less severe side effects, and/or has improved efficacy.

The disclosures of all publications, patents, and patent applications referred to herein are each hereby incorporated herein by reference in their entireties.

### SUMMARY OF THE INVENTION

Described here are novel chimeric molecules which comprise an IL-12 agonist molecule, which comprises an IL-12 p40 subunit (p40), an IL-12 p35 subunit (p35), a masking moiety (MM), and a carrier (C). In some embodiment, said chimeric molecule comprises a p40 mutein, wherein said p40 subunit comprises a mutation, mutations, or a deletion of one or more amino acids. In some embodiment, said chimeric molecule comprises a p35 mutein, wherein said p35 subunit comprises one or more point mutations. In some embodiment, both the p40 and the p35 subunits of the chimeric molecules contain and/or have a mutation or mutations. In some embodiments, said mutation or mutations in the p40 subunit and/or the p35 subunit reduce a biological activity of said chimeric molecule compared to the chimeric molecule with the wild type p40 and p35 subunits. In some embodiments, said p40 subunit comprises the mutations Lys260Ala and Arg261Ala (numbering according to SEQ ID NO: 5). In some embodiments, said p35 subunit comprises a mutation at Tyr40 (numbering according to SEQ ID NO: 6). In an embodiment, p35 subunit comprises the mutation Tyr40Ala (numbering according to SEQ ID NO: 6).

In an aspect, a chimeric molecule comprises a masking moiety (MM) which inhibits the binding of the IL-12 cytokine moiety with an IL-12 receptor β1 subunit, while said p35 subunit comprises a mutation or mutations which reduce the binding affinity of the cytokine moiety with the IL-12 receptor β2 subunit. In another embodiment, said p35 subunit comprises a mutation at Tyr40 (numbering according to SEQ ID NO: 6). In an embodiment, said p35 subunit comprises the mutation Tyr40Ala (numbering according to SEQ ID NO: 6).

In an aspect, a chimeric molecule is a prodrug, which is/are activatable at the site of a tumor, in the tumor microenvironment, or near a tumor. In some embodiments, the carrier moiety of said chimeric molecule comprises an antigen-binding moiety, which binds to an antigen expressed on an immune cell. In another embodiment, an immune cell is infiltered into a tumor or in the tumor microenvironment or locates near a tumor. In some embodiment, said chimeric molecule has a higher activity stimulating an immune cell expressing said antigen targeted by the antigen-binding moiety compared to an immune cell without expressing said antigen. In some embodiment, the EC50 of the chimeric molecule stimulating an immune cell expressing the targeted antigen is at least two times smaller, at least 5 times smaller, or at least 10 times smaller compared to the EC50 of said chimeric molecule stimulating an immune cell without expressing said antigen. In another embodiment, said chimeric molecule is activated even though the chimeric molecule does not comprise a cleavable peptide linker or it comprises a cleavable peptide linker but said cleavable linker is not cleaved. In some embodiments, said chimeric molecule: (a) comprises an IL-12 cytokine moiety, a masking moiety, and a carrier which comprises an antigen-binding moiety which binds to antigen expressed on an immune cell; (b) does not comprises any cleavable peptide linker; and (c) is able to activate an immune cell which expresses an antigen targeted by the carrier. In some embodiment, said antigen is selected from PD-1, TIM-3, TIGIT, LAG-3, CD16A, NKG2A, NKG2D, KIR, SIRPalpha, and NKp46. In some embodiments, said carrier comprises an antigen-binding moiety which binds to PD-1; wherein said antigen-binding moiety comprises the same heavy chain variable domain (VH) and the same light chain variable domain (VL) as that of pembrolizumab or nivolumab. In some embodiments, said VH domain comprises an amino acid sequence of SEQ ID NO: 101 or at least 90% identical as that of SEQ ID NO: 101, and said VL domain comprises an amino acid sequence of SEQ ID NO: 102 or at least 90% identical as that of SEQ ID NO: 102. In an embodiment, said carrier comprises an antigen-binding moiety which binds to LAG-3; wherein said LAG-3-binding moiety comprises an amino acid sequence selected from SEQ ID NOs: 69-77, or at least 90% identical to one selected from SEQ ID NOs: 69-77.

In another embodiment, said chimeric molecule comprises an IL-12 p40 subunit (p40), an IL-12 p35 subunit (p35), a masking moiety (MM), a carrier (C), and a cleavable peptide linker which is cleavable by a protease preferentially expressed at the site of a tumor, in the tumor microenvironment, or near a tumor. In some embodiment, said carrier comprises an Fc domain of an IgG; wherein said masking moiety is linked directly or indirectly to the carrier through a non-cleavable peptide linker; and wherein said p40 subunit of IL-12 is linked to the carrier directly or indirectly through a cleavable peptide linker. In some embodiment, said carrier comprises an antigen-binding moiety; wherein said masking moiety is linked directly or indirectly to the carrier through a cleavable peptide linker; and wherein said p40 subunit of IL-12 is linked to the carrier directly or indirectly optionally through a non-cleavable peptide linker.

In an aspect, a chimeric molecule comprises an IL-12 p40 subunit (p40), an IL-12 p35 subunit (p35), a masking moiety (MM), and a carrier (C); wherein said masking moiety is directly fused to the carrier optionally through a cleavable or non-cleavable peptide linker; wherein said p40 subunit is directly fused to the carrier optionally through a cleavable or non-cleavable peptide linker; and wherein said p35 subunit is fused to the p40 subunit through a peptide linker of 3, 4, 5, 6, 7, 8, 9, or 10 amino acids. In some embodiment, said p35 subunit is fused to the p40 subunit through a peptide linker comprising GGGGS or GGGGSGGGGS.

In another aspect, a chimeric molecule comprises an IL-12 p40 subunit (p40), an IL-12 p35 subunit (p35), a masking moiety (MM), and a carrier (C); wherein:
a. said IL-12 subunit p40 has an amino acid sequence SEQ ID NO: 5 or at least 95% identical to that of SEQ ID NO: 5;
b. said IL-12 subunit p35 has an amino acid sequence SEQ ID NO: 6 or at least 95% identical to that of SEQ ID NO: 6;
c. said masking moiety comprises an amino acid sequence of SEQ ID NO: 1, 2, 3 or 4 or at least 95% identical to that of SEQ ID NO: 1, 2, 3 or 4;
d. said carrier is selected from an albumin or albumin fragment, an Fc domain, and an antibody; and
e. said IL-12 p40 subunit comprises one or more mutations at the site or sites selected from Lys258, Ser259, Lys260, Arg261, Lys263, Lys264, and Asp265, and Arg266 (numbering according to SEQ ID NO: 5).

In some aspect, a chimeric molecule comprises an IL-12 p40 subunit (p40), an IL-12 p35 subunit (p35), a masking moiety (MM), and a carrier (C); wherein:
a. said IL-12 subunit p40 has an amino acid sequence SEQ ID NO: 5 or at least 95% identical to that of SEQ ID NO: 5;
b. said IL-12 subunit p35 has an amino acid sequence SEQ ID NO: 6 or at least 95% identical to that of SEQ ID NO: 6;
c. said masking moiety comprises an amino acid sequence of SEQ ID NO: 1, 2, 3 or 4 or at least 95% identical to that of SEQ ID NO: 1, 2, 3 or 4;
d. said carrier is selected from an albumin or albumin fragment, an Fc domain, and an antibody; and
e. said IL-12 p40 subunit comprises mutations Lys260Ala and Arg261Ala (numbering according to SEQ ID NO: 5).

In another aspect, a chimeric molecule comprises an IL-12 p40 subunit (p40), an IL-12 p35 subunit (p35), a masking moiety (MM), and a carrier (C); wherein:
a. said IL-12 subunit p40 has an amino acid sequence SEQ ID NO: 46, 63, or 98 or at least 99% identical to that of SEQ ID NO: 46, 63, or 98;
b. said IL-12 subunit p35 has an amino acid sequence SEQ ID NO: 6 or at least 95% identical to that of SEQ ID NO: 6;
c. said masking moiety comprises an amino acid sequence of SEQ ID NO: 1, 2, 3 or 4 or at least 95% identical to that of SEQ ID NO: 1, 2, 3 or 4; and
d. said carrier is selected from an albumin or albumin fragment, an Fc domain, and an antibody.

In an embodiment, a chimeric molecule is one selected from any of the above, wherein:
a. said masking moiety is linked to the carrier through a cleavable linker, a non-cleavable linker or without a peptide linker;
b. said p40 subunit is fused to the carrier through a non-cleavable linker or without a peptide linker; and
c. said p35 subunit is fused to the p40 subunit through a non-cleavable linker or without a peptide linker.

In some embodiments, said chimeric molecule comprises a first polypeptide chain and a second polypeptide chain; wherein a first polypeptide chain comprises an amino acid sequence of SEQ ID NO: 80 or at least 90% identical as that of SEQ ID NO: 80, and a second polypeptide chain comprises an amino acid sequence of SEQ ID NO: 81 or at least 90% identical as that of SEQ ID NO: 81.

In an embodiment, said chimeric molecule further comprises an antigen-binding moiety which binds to CD40, OX40, GITR, 4-1BB, CD16A, NKG2A, or NKG2D.

In another embodiment, a chimeric molecule further comprises one or more copies of an ectodomain or extracellular domain (ECD) of the CD40 ligand (CD40L), the OX40 ligand (OX40L), the GITR ligand (GITRL), the 4-1 BB ligand (4-1 BBL), a ligand for NKG2D or its analog, CD47, or SIRPα.

In some embodiments, a chimeric molecule further comprises an ectodomain of CD40L, which comprises an amino acid sequence of SEQ ID NO: 7 or at least 90% identical as that of SEQ ID NO: 7.

In an embodiment, a chimeric molecule further comprises an antigen binding moiety, which binds to an antigen expressed on the surfaced of a tumor cell, a cancer cell, or an immune cell.

In an embodiment, a chimeric molecule further comprises an antigen binding moiety, which binds to an antigen selected from PD-1, PD-L1, CD47, CD16a, NKG2D, NKG2A, SIRPα, Siglec-10, TIGIT, TIM-3, LAG-3, 5T4, FAP, EGFR, CMET, HER2, Trop-2, VEGFR2, ROR1, mesothelin, GPC-3, CD20, CD38, and BCMA.

In another embodiment, a chimeric molecule further comprises an antigen binding moiety, which binds to LAG-3; wherein said LAG-3-binding moiety is a single domain antibody, which comprises an amino acid sequence selected from SEQ ID NOs: 69-77, or at least 90% identical to one selected from SEQ ID NOs: 69-77.

In some embodiments, a chimeric molecule further comprises an antigen binding moiety, which binds to a PD-1; wherein said PD-1-binding moiety is an antibody or a binding fragment thereof, which comprises a heavy chain variable domain (VH) with an amino acid sequence of SEQ ID NO: 101 or at least 95% identical as that of SEQ ID NO: 101, and a light chain variable domain (VL) with an amino acid sequence of SEQ ID NO: 102 or at least 95% identical as that of SEQ ID NO: 102.

In an embodiment, a p35 subunit of any of above said chimeric molecule comprises one or more mutations selected from K128A, F166K, E38A, F39A, Y40A, T43A, S44G, E45A, E46A, D48A, H49A, E50A, E79A, T80A, F82A, and N71Q (numbering according to SEQ ID NO: 6). In some embodiment, said p35 subunit comprises double mutations selected from E38A/F39A, T43A/S44G, E45A/E46A, D48A/H49A, and E79A/T80A (numbering according to SEQ ID NO: 6).

In another aspect, a chimeric molecule comprises an IL-12 p40 subunit (p40), an IL-12 p35 subunit (p35), and a carrier (C); wherein said p35 comprises one or more mutations selected from K128A, F166K, T36A, L37E, E38A, F39A, E38A, F39A, Y40A, P41G, T43A, S44G, E45A, E46A, D48A, H49A, E50A, E79A, T80A, F82A, and N71Q (numbering according to SEQ ID NO: 6); and wherein said carrier is selected from an albumin or albumin fragment, an Fc domain, and an antibody or a binding fragment thereof.

In an aspect, a chimeric molecule comprises an IL-12 p40 subunit (p40), an IL-12 p35 subunit (p35), and a carrier (C); wherein said p35 comprises double mutations selected from E38A/F39A, T43A/S44G, E45A/E46A, D48A/H49A, and E79A/T80A (numbering according to SEQ ID NO: 6); and wherein said carrier is selected from an albumin or albumin fragment, an Fc domain, and an antibody or a binding fragment thereof.

In some embodiments, a chimeric molecule further comprises a masking moiety (MM); wherein: (a) an IL-12 subunit p40 has an amino acid sequence SEQ ID NO: 5 or at least 95% identical to that of SEQ ID NO: 5; and (b) a masking moiety comprises a binding moiety comprising an amino acid sequence of SEQ ID NO: 1, 2, 3 or 4 or at least 95% identical to that of SEQ ID NO: 1, 2, 3 or 4. In an embodiment, a chimeric molecule further comprises a masking moiety (MM); wherein: (a) a masking moiety is linked to the carrier through a cleavable linker, a non-cleavable linker or without a peptide linker; (b) a p40 subunit is fused to the carrier through a cleavable peptide linker, a non-cleavable peptide linker or without a peptide linker; and (c) a p35 subunit is fused to the p40 subunit through a non-cleavable linker or without a peptide linker.

In another embodiment, a carrier comprises an antigen-binding moiety, which binds to an antigen selected from PD-1, PD-L1, CD47, CD16a, NKG2D, SIRPα, Siglet-10, TIGIT, TIM-3, LAG-3, 5T4, FAP, EGFR, CMET, HER2, Trop-2, VEGFR2, ROR1, mesothelin, GPC-3, CD20, CD38, and BCMA.

In an embodiment, a carrier comprises an antigen-binding moiety, which binds to LAG-3; wherein said LAG-3-binding moiety is a single domain antibody, which comprises an amino acid sequence selected from SEQ ID NOs: 69-77, or at least 90% identical to one selected from SEQ ID NOs: 69-77.

In another embodiment, a carrier comprises an antigen-binding moiety, which binds to PD-1; wherein a PD-1-binding moiety is an antibody or a binding fragment thereof, which comprises a VH domain with an amino acid sequence of SEQ ID NO: 101 or at least 95% identical as that of SEQ ID NO: 101, and a VL domain an amino acid sequence of SEQ ID NO: 102 or at least 95% identical as that of SEQ ID NO: 102.

In some embodiments, a carrier comprises an antigen-binding moiety, which binds to 5T4; wherein said 5T4-binding moiety is an antibody or a binding fragment thereof, which comprises a VH domain with an amino acid sequence of SEQ ID NO: 78 or at least 95% identical as that of SEQ ID NO: 78, and a VL domain an amino acid sequence of SEQ ID NO: 79 or at least 95% identical as that of SEQ ID NO: 79.

A chimeric molecule, which comprises a first polypeptide chain and a second polypeptide chain; wherein a first polypeptide chain comprises an amino acid sequence of SEQ ID NO: 97 or an amino acid sequence at least 99% identical as that of SEQ ID NO: 97; and wherein a second polypeptide chain comprises an amino acid sequence selected from SEQ ID NOs: 82-96, and 99 or an amino acid sequence at least 99% identical as one selected from SEQ ID NOs: 82-96, and 99.

In some embodiment, said chimeric molecule comprises two or more polypeptide chains; wherein one of its polypeptide chains comprises an amino acid sequence selected from SEQ ID NOs: 82-96, or an amino acid sequence at least 95% identical as one selected from SEQ ID NOs: 82-96.

In some embodiment, said chimeric molecule further comprises a masking moiety, wherein said masking moiety comprises an amino acid sequence selected from SEQ ID NOs: 1-4 or at least 90% identical as that of SEQ ID NO: 1-4.

In some embodiment, said chimeric molecule further comprises an antigen-binding moiety; wherein an antigen-binding moiety binds to an antigen selected from PD-1, PD-L1, CD47, CD16a, NKG2D, NKG2A, SIRPα, Siglec-10, TIGIT, TIM-3, LAG-3, 5T4, FAP, EGFR, CMET, HER2, Trop-2, VEGFR2, ROR1, mesothelin, GPC-3, CD20, CD38, and BCMA.

In some embodiments, a chimeric molecule binds to LAG-3 and comprises two polypeptide chains; wherein a first polypeptide chain comprises an amino acid sequence selected from SEQ ID NOs: 111-114 or at least 99% identical as one selected from SEQ ID NOs: 111-114; and wherein a second polypeptide chain comprises an amino acid sequence selected from SEQ ID NOs: 115-124 or at least 99% identical as one selected from SEQ ID NO: 115-124. In some embodiment, said chimeric molecule selectively stimulates a LAG-3 positive (LAG-3⁺) immune cell with significantly higher activity than stimulating a LAG-3⁻ immune cell.

In some aspect, a chimeric molecule comprises an IL-12 p40 subunit (p40), an IL-12 p35 subunit (p35), a masking moiety (MM), and a carrier (C); wherein:
a. said IL-12 subunit p40 comprising an amino acid sequence of SEQ ID NO: 5 or an amino acid sequence of at least 95% identity to that of SEQ ID NO: 5;
b. said IL-12 subunit p35 comprising an amino acid sequence of SEQ ID NO: 6 or an amino acid sequence of at least 95% identity to that of SEQ ID NO: 6;
c. said masking moiety comprises an amino acid sequence of SEQ ID NOS: 1, 2, 3 or 4, wherein the amino acid sequence has a homology of at least 95% identity to that of SEQ ID NO: 1, 2, 3 or 4;
d. said carrier comprising an antigen-binding moiety which binds to PD-1 and an Fc domain with a first Fc polypeptide and a second Fc polypeptide;
e. said masking moiety is fused to the C-terminus of the first Fc polypeptide with or without a non-cleavable or cleavable peptide linker; wherein said peptide linker optionally comprises an amino acid sequence of 3 amino acids to 22 amino acids in length;
f. said p40 subunit is fused to the 2^{nd} Fc polypeptide optionally through a non-cleavable peptide linker; and
g. said p35 subunit is fused to the C-terminus of the p40 subunit.

In some embodiment, said chimeric molecule has higher activity stimulating a PD-1 positive (PD-1⁺) T compared to that of stimulating an NK cell.

In some embodiment, said masking moiety is fused to C-terminus of the first Fc polypeptide with a non-cleavable peptide linker; wherein said non-cleavable peptide linker optionally comprises an amino acid sequence of 3 amino acids to 22 amino acids in length; and wherein said chimeric molecule has higher activity stimulating a PD-1V immune cell than that of stimulating a PD-1 negative (PD-1⁻) immune cell.

In some embodiment, said p35 subunit is fused to the p40 subunit with a non-cleavable peptide linker of 2-10 amino acids in length, which optionally comprising an amino acid sequence selected from GGS, GGGGS, GGSGGSGGS, or GGGGSGGGGS.

In some embodiment, said chimeric molecule comprises two polypeptide chains; wherein said first polypeptide chain comprises an amino acid sequence selected from SEQ ID NOs: 103-108 or an amino acid sequence of at least 99% identity to one of SEQ ID NOs: 103-108; and wherein said second polypeptide chain comprises an amino acid sequence selected from SEQ ID NOs: 109 and 110 or an amino acid sequence of at least 99% identity to one of SEQ ID NOs: 109 or 110.

In some embodiment, said chimeric molecule comprises two identical light chains and a first heavy chain polypeptide chain and a second heavy chain polypeptide chain; wherein said light chains comprise an amino acid sequence of SEQ ID NO: 129 or an amino acid sequence of at least 95% identity to SEQ ID NO: 129; further wherein said first heavy chain polypeptide chain comprises an amino acid sequence selected from SEQ ID NOs: 125-127 or an amino acid sequence of at least 99% identity to SEQ ID NOs: 125-127; and further wherein said second heavy chain polypeptide chain comprises an amino acid sequence as SEQ ID NO: 128 or an amino acid sequence of at least 99% identity to SEQ ID NO: 128.

In some embodiment, said chimeric molecule comprises two identical light chains and a first heavy chain polypeptide chain, and a second heavy chain polypeptide chain; wherein said light chains comprise an amino acid sequence of SEQ ID NO: 129 or an amino acid sequence of at least 95% identity to SEQ ID NO: 129; wherein said first heavy chain polypeptide chain comprises an amino acid sequence selected from SEQ ID NOs: 130-135 or an amino acid sequence of at least 99% identity to one of SEQ ID NOs: 130-135; and further wherein said second heavy chain polypeptide chain comprises an amino acid sequence of SEQ ID NO: 136 or an amino acid sequence of at least 99% identity to one of SEQ ID NO: 136.

In some embodiment, said chimeric molecule stimulates a PD-1⁺ immune cell, wherein the stimulation results in a significantly higher activity than that of a PD-1⁻ immune cell; and wherein the EC50 for stimulating said PD-1⁻ immune cell is at least 2 times greater, at least 5 times greater, or at least 10 times greater than that of the PD-1⁺ immune cell.

In some embodiment, said chimeric molecule has significantly higher activity stimulating a PD-1⁺ T cell expressing and that stimulating an NK cell; wherein the EC50 for stimulating said NK cell is at least 2 times greater, at least 5 times greater, or at least 10 times greater than that of said T cells.

Also disclosed is a pharmaceutical composition comprising any of the above said chimeric molecules or prodrugs.

Also presented is a polynucleotide or polynucleotides encoding any one of the above said chimeric molecules or prodrugs.

Also presented is an expression vector or vectors comprising the above said polynucleotide or polynucleotides.

Also disclosed is a method of making an antigen-binding molecule of any one of the chimeric molecules, comprising culturing an above said host cell under conditions that allow expression of said chimeric molecule, and isolating said chimeric molecule.

Also disclosed is a method of treating a cancer or an infectious disease or stimulating the immune system in a patient in need thereof, comprising administering the above said pharmaceutical composition.

Also presented is a chimeric molecule for use in treating a cancer or an infectious disease or stimulating the immune system in a method disclosed herein. In some embodiment, a cancer is selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.

In an embodiment, a method of treating of a patient with cancer, comprises administering said pharmaceutical composition of the present invention directly into a tumor or tumors.

### BRIEF DESCRIPTION OF THE DRAWINGS

FIGS. 1A, 1B, 1C, and 1D show illustrations of the structure of an IL-12 chimeric molecule comprising an IL-12 p40 subunit (p40), an IL-12 p35 subunit (p35), a masking moiety, and an Fc domain. FIGS. 1A and 1B show the IL-12 subunits and the masking moiety fused to the C-termini the Fc domain polypeptide chains. FIGS. 1C and 1D show the IL-12 subunits and the masking moiety fused to the N-termini the Fc domain polypeptide chains.

FIGS. 2A, 2B, 2C, and 2D show illustrations of the structure of an IL-12 chimeric molecule comprising an IL-12 p40 subunit (p40), an IL-12 p35 subunit (p35), a first masking moiety, a second masking moiety and an Fc domain. FIGS. 2A and 2B show the IL-12 subunits and the masking moieties fused to the C-termini the Fc domain polypeptide chains. FIGS. 2C and 2D show the IL-12 subunits and the masking moieties fused to the N-termini the Fc domain polypeptide chains.

FIGS. 3A, 3B, 3C, and 3D show illustrations of the structure of an IL-12 chimeric molecule comprising an IL-12 p40 subunit (p40), an IL-12 p35 subunit (p35), a masking moiety, an Fc domain, and 2-6 copies of CD40L ectodomain. FIGS. 3A and 3B show the IL-12 subunits and the masking moiety fused to the C-termini the Fc domain polypeptide chains; and the CD40L ectodomains are fused to the N-termini of the Fc domain. FIGS. 3C and 3D show the IL-12 subunits and the masking moiety fused to the N-termini the Fc domain polypeptide chains; and the CD40L ectodomains are fused to the C-termini of the Fc domain.

FIGS. 4A, 4B, 4C, and 4D show illustrations of the structure of an IL-12 chimeric molecule comprising an IL-12 p40 subunit (p40), an IL-12 p35 subunit (p35), a first masking moiety, a second masking moiety, an Fc domain, and 2-6 copies of CD40L ectodomains. FIGS. 4A and 4B show the IL-12 subunits and the masking moiety fused to the C-termini the Fc domain polypeptide chains; and the CD40L ectodomains are fused to the N-termini of the Fc domain. FIGS. 4C and 4D show the IL-12 subunits and the masking moiety fused to the N-termini the Fc domain polypeptide chains; and the CD40L ectodomains are fused to the C-termini of the Fc domain.

FIGS. 5A, 5B, 5C, and 5D show illustrations of the structure of an IL-12 chimeric molecule comprising an IL-12 p40 subunit (p40), an IL-12 p35 subunit (p35), a masking moiety, an Fc domain, 1-3 copies of CD40L ectodomain, and an antigen-binding moiety. FIGS. 5A and 5B show the IL-12 subunits and the masking moiety fused to the C-termini the Fc domain polypeptide chains; and the CD40L ectodomains and the antigen-binding moiety are fused to the N-termini of the Fc domain. FIGS. 5C and 5D show the IL-12 subunits and the masking moiety fused to the N-termini the Fc domain polypeptide chains; and the CD40L ectodomains and the antigen-binding moiety are fused to the C-termini of the Fc domain.

FIGS. 6A, 6B, 6C, and 6D show illustrations of the structure of an IL-12 chimeric molecule comprising an IL-12 p40 subunit (p40), an IL-12 p35 subunit (p35), a first masking moiety, a second masking moiety, an Fc domain, and 2-6 copies of CD40L ectodomains. FIGS. 6A and 6B show the IL-12 subunits and the masking moiety fused to the C-termini the Fc domain polypeptide chains; and the CD40L ectodomains and the antigen-binding moiety are fused to the N-termini of the Fc domain. FIGS. 6C and 6D show the IL-12 subunits and the masking moiety fused to the N-termini the Fc domain polypeptide chains; and the CD40L ectodomains and the antigen-binding moiety are fused to the C-termini of the Fc domain.

FIG. 7A shows the results of the NK92 interferon gamma releasing assay. The samples include chimeric molecules IL12p35-y40A, IL12p35-F166K, IL12p40-deletion, IL-12p40-AA, IL-12p35-N71Q and IL-12p35-T43A/S44G which comprise mutation or mutations in the IL-12 cytokine moiety. Chimeric molecule “IL-12 wt U mask” comprises the wild type IL-12. All those chimeric molecules are IL-12 prodrugs. The protease activated versions of the prodrugs were also included in the assay. The chimeric molecules after activation are named IL12p35-y40A act, IL12p35-F166K act, IL12p40-deletion act, IL-12p40-AA act, IL-12p35-N71Q act, IL-12p35-T43A/S44G act, and IL-12 wt U mask act, respectively.

FIG. 7B shows the amino acid sequences of the samples.

FIG. 8 illustrates the structure of a chimeric molecule which comprises a single domain antibody against LAG-3, which further comprises an Fc domain, an IL-12 p40 subunit polypeptide, an IL-12 p35 subunit polypeptide, and a masking moiety. Briefly, the single domain antibody is fused to the N-terminus of each of the Fc polypeptide through a non-cleavable peptide linker, said masking moiety is fused to the C-terminus of one of the Fc polypeptide chains, and said p40 subunit is fused to the C-terminus of the other Fc polypeptide chain; wherein said p35 is fused to the C-terminus of the p40 subunit. In an embodiment, said chimeric molecule does not comprise any cleavable peptide linker.

FIG. 9A illustrates the structure of a chimeric molecule which comprises an scFv against PD-1, which further comprises an Fc domain, an IL-12 p40 subunit polypeptide, an IL-12 p35 subunit polypeptide, and a masking moiety. Briefly, the scFv is fused to the N-terminus of each of the Fc polypeptide through a non-cleavable peptide linker, said masking moiety is fused to the C-terminus of one of the Fc polypeptide chains, and said p40 subunit is fused to the C-terminus of the other Fc polypeptide chain; wherein said p35 is fused to the C-terminus of the p40 subunit. In another embodiment, said chimeric molecule does not comprise any cleavable peptide linker.

FIG. 9B illustrates the structure of a chimeric molecule which comprises an antibody against PD-1, which further comprises an IL-12 p40 subunit polypeptide, an IL-12 p35 subunit polypeptide, and a masking moiety. Briefly, said masking moiety is fused to the C-terminus of one of the heavy chain polypeptide chains, and said p40 subunit is fused to the C-terminus of the other heavy chain polypeptide chain; wherein said p35 is fused to the C-terminus of the p40 subunit. In another embodiment, said chimeric molecule does not comprise any cleavable peptide linker.

FIG. 10A shows the results of the interferon gamma (IFNγ) releasing assay with chimeric molecules which comprise Fc domain as the carrier.

FIG. 10B shows the descriptions of the samples and the corresponding EC50 values. The samples comprise masking moieties derived from anti-IL-12 antibodies briakinumab (Bmask) or ustekinumab (Umask). The cytokine moiety comprises wild type IL-12 or a mutant IL-12 polypeptide selected from p35-Y40A and p35-F166A. The results showed that both Bmask and Umask inhibited the activities of IL-12. In addition, p35-Y40A mutation further reduced the activity of the chimeric molecule.

FIG. 11 shows the results of the HEK Blue-IL12 assay for the purified chimeric molecules ASKG129A, ASKG129B and ASKG129C. The data showed that all three molecules had significantly reduced biological activities as compared with IL-12. EC50 values of the molecules were over 200 times to 5000 times larger than IL-12. Notably, the masking moiety and the p35-Y40A mutation together significantly reduced the activities of IL-12.

FIG. 12 shows the cytokine releasing assay with human PBMC. The activity of ASKG129C was reduced by approximately 1000 times compared to that of IL-12.

FIGS. 13A-13G show the results of the ex vivo pSTAT4 analysis. Human recombinant IL-12 induced dose-dependent phosphorylation of STAT4 (p-STAT4) in human PBMC (EC50 values CD4⁺ T cells: 0.072 nM, CD8⁺ T cells: 0.045 nM, NK cells: 0.1662 nM), compared with Lag3-129C (or ASKG129C) (EC50 values CD4⁺ T cells: 2.52 nM, CD8⁺ T cells: 2.09 nM, NK cells: 3.75 nM). The E-max (Maximal response) for human IL-12-induced CD8⁺ T cells and NK cells was around 60 to 80% p-STAT4 positive cells vs 40 to 50% p-STAT4 positive CD4⁺ T cells. However, the Lag3-129C (or ASKG129C)-induced Emax response was much reduced in CD8⁺ T cells, NK cells, and CD4⁺ T cells (30%, 20%, and 10%) respectively. The activity of Lag3-129C to induce activation of STAT4 was significantly reduced compared with human recombinant IL-12 in CD4⁺ T cells, CD8⁺ T cells and NK cells.

FIG. 14 shows the purity analysis of the chimeric molecules by reduced and non-reduced SDS-PAGE. The amino acid sequence information of the molecules is shown in Table 8.

FIG. 15 shows the HEK-Blue IL-12 Assay results for the chimeric molecule SR2.7.4 which comprises an anti-PD-1 antibody and an IL-12 mutant, wherein said mutant comprises p40-K260A/R261A mutations. The activity of said chimeric molecule was lower compared to the Fc-based IL-12 chimeric molecule LL25-18, which comprises the wild type IL-12 and is masked by the U mask. In addition, both molecules had lower activity compared to the reference molecule, which is an IL-12 mutant molecule fused to the Fc domain, which was disclosed in United States Patent Application 20200216509.

FIGS. 16A, 16B and 16C show the SEC-HPLC chromatographs for purified ASKG129A (Lot #LL25-55), ASKG12B (Lot #LL25-58E2), and ASKG12C (Lot #LL25-52). All the purified samples had SEC-HPLC main peak purities of approximately 95% or higher.

### DETAILED DESCRIPTION OF THE INVENTION

As used herein and in the appended claims, the singular forms “a,” “or,” and “the” include plural referents unless the context clearly dictates otherwise. Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The terms “cytokine agonist polypeptide” or “cytokine moiety” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine mask” or “masking moiety” refers to a moiety (e.g., a polypeptide) that binds to a cytokine, thereby inhibiting the cytokine from binding to its receptor on the surface of a target cell and/or exerting its biological functions while being bound by the mask. Examples of a cytokine mask include, without limitations, a polypeptide derived from an extracellular domain of the cytokine's natural receptor that makes contact with the cytokine. Additional examples of a cytokine mask include, without limitations, an antibody or a binding fragment thereof or a single domain antibody which binds to the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to an animal, including, but not limited to, a mammal and further includes, but is not limited to, a human, a pet (e.g., a canine or a feline), a farm animal (e.g., cattle or horse), a rodent, or a primate. Animals also include, but are not limited to, a chicken, a turkey, a donkey, an ostrich, a human, a monkey, a rat, a mouse, a goat, a sheep, a pig, an emu, or an alpaca.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### I. Chimeric Molecules Comprising an IL-12 Agonist Polypeptide

Described herein are novel chimeric molecules which comprise an IL-12 agonist molecule. In some embodiments, a chimeric molecules are prodrugs, which are optionally activatable at the site of a tumor, even though it does not comprise any cleavable peptide linker.

The IL-12 chimeric molecules or prodrugs have fewer side effects, better in vivo PK profiles (e.g., longer half-life) and better target specificity, and are more efficacious as compared to prior IL-12 therapeutics. The present IL-12 chimeric molecules or prodrugs comprise an IL-12 agonist molecule, one or two masking moieties (M), a carrier (C), optionally 1-6 copies of the ectodomains of CD40L, OX40L, 4-1 BBL or GITRL, and optionally an antigen-binding moiety.

IL-12 comprises subunits p40 and p35. Subunit p40 comprises an amino acid as shown in SEQ ID NO: 5 and p35 comprises an amino acid sequence as shown in SEQ ID NO: 6. In some embodiments, said IL-12 analog comprises subunit p40 with an amino acid that is at least 90% identical to SEQ ID NO: 5. In some embodiments, said IL-12 analog comprises subunit p40 with an amino acid of SEQ ID NO: 46 or 63. In other embodiments, said IL-12 analog comprises subunit p35 with an amino acid sequence that is at least 90% identical to SEQ ID NO: 6.

In some embodiment, IL-12 subunit p40 comprises one or more mutation(s) or deletion(s) at a site or sites selected from Lys258, Ser259, Lys260, Arg261, Lys263, Lys264, and Asp265, and Arg266 (numbering according to SEQ ID NO: 5). In another embodiment, the IL-12 P40 subunit comprises mutations Lys260Ala and Arg261Ala (numbering according to SEQ ID NO: 5). In an embodiment, the IL-12 P40 subunit comprises an amino acid sequence of SEQ ID NO: 98 or at least 95% identical as that of SEQ ID NO: 98.

In some embodiment, said p35 subunit comprises one or more mutations selected from K128A, F166K, E38A, F39A, Y40A, T43A, S44G, E45A, E46A, D48A, H49A, E50A, E79A, T80A, F82A, and N71Q (numbering according to SEQ ID NO: 6).

In some embodiment, said p35 subunit comprises one or more mutations selected from K128A, F166K, E38A, F39A, Y40A, T43A, S44G, E45A, E46A, D48A, H49A, E50A, E79A, T80A, F82A, and N71Q (numbering according to SEQ ID NO: 6).

In some embodiments, said p35 subunit comprises a mutation or mutations selected from Y40A, T43A/S44G, N71Q, and F166K.

In another embodiment, said chimeric molecule comprises one or more polypeptide chains; wherein one of a polypeptide chains comprises an amino acid sequence selected from SEQ ID NOs: 82-96, and 99, or an amino acid sequence at least 90%, at least 95%, or at least 99% identical as one selected from SEQ ID NOs: 82-96, and 99.

In further embodiment, said chimeric molecule comprises an IL-12 p40 subunit (p40) and an IL-12 p35 subunit (p35); wherein said p35 comprises one or more mutations selected from K128A, F166K, E38A, F39A, Y40A, T43A, S44G, E45A, E46A, D48A, H49A, E50A, E79A, T80A, F82A, and N71Q (numbering according to SEQ ID NO: 6).

In an aspect, a chimeric molecule comprises an IL-12 p40 subunit (p40) and an IL-12 p35 subunit (p35); wherein said p35 comprises double mutations selected from E38A/F39A, T43A/S44G, E45A/E46A, D48A/H49A, and E79A/T80A (numbering according to SEQ ID NO: 6).

In a further embodiment, the IL-12 chimeric molecule/prodrugs comprise at least one masking moiety. The masking moieties may be linked to the cytokine moiety or to the carrier moiety through a peptide linker. In some embodiments, the peptide linker is a non-cleavable peptide linker. The mask inhibits the cytokine moiety's biological functions while the mask is binding to it. In specific embodiments, the mask inhibits a biological activity of IL-12 or its analog.

In some embodiments, the carriers for the IL-12 chimeric molecules or prodrugs are antigen-binding moieties that bind an antigen at a target site (e.g., tumor surface). In some embodiments, the present IL-12 chimeric molecules or prodrugs become active in the body at a target site targeted by the carrier. In further embodiments, the carrier in the prodrug is an antibody targeting a tumor antigen such that the IL-12 prodrug is delivered to a tumor site in a patient.

In some embodiments, the carrier is selected from an albumin, a Fc fragment, a polyethylene glycol (PEG), or an antibody or antigen-binding fragment thereof.

In some embodiments, the IL-12 chimeric molecules/prodrugs comprise an IL-12 agonist polypeptide, a first masking moiety (MM1), a second masking moiety (MM2), a carrier (C), and one or more non-cleavable peptide linkers that link the masking moieties to the agonist polypeptide or the carrier. In some embodiment, said IL-12 chimeric molecules/prodrugs comprise 2-6 copies of ectodomains of CD40L. In some embodiment, said IL-12 chimeric molecules/prodrugs comprise an antigen-binding moiety which binds to an antigen expressed on a tumor cell, a cancer cell or an immune cell. In some embodiment, an antigen binding moiety binds to Ig-Like Transcript 2 (ILT2), HLA-G, NKG2D, FAP, 5T4, PD-L1, PD-1, CD-47, or SIRPα. In some embodiment, said antigen-binding moiety comprises a scFv, a Fab or a binding fragment derived from the ILT2 antibody BDN-22.

### A. Masking Moieties of the IL-12 Chimeric Molecules or Prodrugs

In some embodiments, the carrier is selected from an albumin, a Fc fragment, a polyethylene glycol (PEG), or an antibody or antigen-binding fragment thereof.

In some embodiments, the masking moiety for IL-12 is selected from an IL-12 receptor beta1 subunit extracellular domain or a fragment thereof, an IL-12 receptor beta2 subunit extracellular domain or a fragment thereof, and a scFv or Fab with specificity to p40 or p35. In some embodiments, the masking moiety inhibits a biological activity of IL-12 or its analog. In some embodiments, the masking moiety comprises a scFv, wherein the scFv has an amino acid sequence at least 90%, at least 95%, or at least 99% identical to one selected from SEQ ID NOs: 1-4.

In some embodiment, a chimeric molecule comprises Mask A and Mask B; a Mask A comprises a scFv with an amino acid sequence of SEQ ID NO: 1 or 2 or at least 95% identical as that of SEQ ID NO: 1 or 2; and a Mask B comprises a scFv with an amino acid sequence of SEQ ID NO: 3 or 4 or at least 95% identical as that of SEQ ID NO: 3 or 4; and wherein a chimeric molecule comprises a structure as illustrated in FIG. 2A, 2B, 2C, 2D, 4A, 4B, 4C, 4D, 6A, 6B, 6C, or 6D. In some embodiments, the masking moiety is an IL-12 receptor extracellular domain (ECD) or its functional analog, wherein the IL-12 receptor is IL-12 receptor beta1. In some embodiments, the masking moiety is IL-12 receptor extracellular domain (ECD), its functional analog, or a fragment thereof, wherein the IL-12 receptor is IL-12 receptor beta1. In some embodiments, the prodrug comprises two masking moieties Mask A and Mask B; wherein at least one of the masking moieties comprises a scFv or Fab, which binds to p40 or p35, and reduces or inhibits the binding of IL-12 to IL-12Rβ1 or IL-12Rβ2. In some embodiments, the scFv or Fab comprises the same light chain CDRs and heavy chain CDRs as IL-12. In some embodiment, a Mask A comprises a scFv derived from ustekinumab and a MaskB comprises a scFv derived from briakinumab; and wherein a chimeric molecule comprises a structure as illustrated in FIG. 2A, 2B, 2C, 2D, 4A, 4B, 4C, 4D, 6A, 6B, 6C, or 6D. In some embodiments, the scFv or Fab comprises the same light chain CDRs and heavy chain CDRs as an IL-12 antibody selected from PMA204, Antibody 1, Antibody 50, Antibody 68, Antibody 80, and Antibody 136 (see U.S. Pat. No. 8,563,697).

In some embodiments, both masking moieties (Mask A and Mask B) are scFvs or Fabs; wherein the first masking moiety binds to p40 and reduces or inhibits the binding of IL-12 to IL-12Rβ1, and the second masking moiety binds to p40 or p35, and reduces or inhibits the binding of IL-12 to IL-12Rβ2. In some embodiments, the first masking moiety comprises the same light chain CDRs and heavy chain CDRs as an IL-12 antibody briakinumab or ustekinumab. In some embodiments, the second masking moiety comprises the same light chain CDRs and heavy chain CDRs as an IL-12 antibody selected from PMA204, Antibody 1, Antibody 50, Antibody 68, Antibody 80, and Antibody 136 (see U.S. Pat. No. 8,563,697).

In some embodiment, an IL-12 chimeric molecules are as illustrated in FIGS. 2A, 2B, 2C, 2D, 4A, 4B, 4C, 4D, 6A, 6B, 6C, and 6D. In some embodiment, a Mask A illustrated in the figures is a scFv comprises an amino acid sequence of SEQ ID NO: 1 or 2; wherein a Mask B illustrated in the figures is a scFv comprises an amino acid sequence of SEQ ID NO: 3 or 4.

### B. Carrier Moieties of the Chimeric Molecules or Prodrugs

The carrier moieties of the present chimeric molecules or prodrugs may be an antigen-binding moiety, or a moiety that is not antigen-binding. The carrier moiety may improve the PK profiles, such as serum half-life, of the cytokine agonist polypeptide and may also target the cytokine agonist polypeptide to a target site in the body, such as a tumor site.

### 1. Non-Antigen-Binding Carrier Moieties

Non-antigen-binding carrier moieties may be used for the present chimeric molecules or prodrugs. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

The carrier moiety of the prodrug may comprise an albumin (e.g., human serum albumin) or a fragment thereof. In some embodiments, the albumin or albumin fragment is about 85% or more, about 90% or more, about 91% or more, about 92% or more, about 93% or more, about 94% or more, about 95% or more, about 96% or more, about 97% or more, about 98% or more, about 99% or more, about 99.5% or more, or about 99.8% or more identical to human serum albumin or a fragment thereof.

In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment.

In some embodiments, the carrier is an antibody Fc fragment. Fc is a dimeric molecule that has two N-terminals and two C-terminals. In some embodiments, the cytokine moiety can be fused to one Fc polypeptide in a dimeric Fc fragment, and the masking moieties can be fused to the 2^{nd} Fc polypeptide. In another embodiment, both the cytokine moiety and the masking moiety are fused to the C-terminal of each polypeptide chain of the dimeric Fc fragment. In some embodiments, both the cytokine moiety and the masking moieties are fused to the N-terminal of each polypeptide chain of the dimeric Fc fragment. In either case, at least one of the masking moieties is fused to the Fc polypeptide directly or indirectly through a cleavable peptide linker.

### 2. Antigen-Binding Moieties

The antigen-binding moiety may be an antibody or an antigen-binding fragment thereof, or an immunoadhesin, or a ligand of a receptor. In some embodiments, the antigen-binding moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide. In some embodiments, the antigen-binding moiety comprises a full-length antibody heavy chain or a full-length antibody light chain. In some embodiments, the antigen-binding moiety includes an antibody heavy chain fragment or an antibody light chain fragment.

In some embodiments, the cytokine moiety is fused to the C-terminus of one of the heavy chains of an antibody, and the cytokine's mask is fused to the C-terminus of the other heavy chain of the antibody through a peptide linker, wherein the two heavy chains optionally contain mutations that allow the specific pairing of the two different heavy chains.

Strategies of forming heterodimers for Fc-fusion polypeptides or bispecific antibodies are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech. (1998) 16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the prodrug comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG, (see Dall'Acqua et al., J Biol Chem. (2006) 281: 23514-24; and Robbie et al., Antimicrob Agents Chemother. (2013) 57(12):6147-53). In further embodiments, the YTE mutations are present in the antibody moiety in addition to the knobs-into-holes mutations. In particular embodiments, the antibody moiety has YTE, LALA and knobs-into-holes mutations or any combination thereof.

In some embodiments, the antibody binds to PD-L1. In some embodiment, the antibody binds to CEA. In some embodiment, the antibody binds to an antigen on a tumor cell, for examples, 5T4, FAP, Trop-2, PD-L1, HER-2, EGFR, Claudin 18.2, DLL-3, GCP3, and CEA. The antibody may or may not have Antibody-Dependent Cellular Cytotoxicity (ADCC) activity. The antibody may also be further conjugated with cytotoxic drugs. In some embodiment, the antibody binds to a target on the surface of an immune cell and has the ability to activate a immune cell and enhance its anti-cancer activity, for examples, ILT-2 antibody, PD-1 antibody, LAG3 antibody, TIGIT antibody, TGF-beta antibody, and CTLA4 antibody.

The antigen-binding moiety can bind an antigen on the surface of a cell, such as a cancer cell. In some embodiments, the antigen-binding moiety is a bispecific antigen-binging moiety, can bind to two different antigens or two different epitopes on the same antigen. In some embodiments, the antigen-binding moiety binds to Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1) 1, vascular endothelial growth factor receptor 2 (VEGFR2), Nectin-4, Liv-1, glycoprotein NMB (GPNMB), prostate-specific membrane antigen (PSMA), Trop-2, carbonic anhydrase IX (CA9), endothelin B receptor (ETBR), Thomsen-Friedenrech antigen (TF), sodium-dependent phosphate transport protein 2B (NaPi2b), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, programmed death ligand 1 (PD-L1), Claudin 18.2, Claudin 6, PSMA, or FAP-alpha. In some embodiments, the antigen-binding moiety binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moiety binds to an epitope located after the 374^{th} amino acid of GPC3. In some embodiments, the antigen-binding moiety binds to a heparin sulfate glycan of GPC3. In some embodiments, the antigen-binding moiety binds to Claudin 18.2 and does not bind to Claudin 18.1. In some embodiments, the antigen-binding moiety binds to Claudin 18.1 with at least 10 times weaker binding affinity than to Claudin 18.2.

The antigen-binding moiety can bind an antigen on the surface of a cell, such as an immune cell, for example T cells, NK cells, and macrophages. In some embodiments, the antigen-binding moiety is a bispecific antigen-binging moiety, can bind to two different antigens or two different epitopes on the same antigen. In some embodiments, the antigen-binding moiety binds to ILT2, PD-1, LAG-3, TIM-3, CTLA-4, or TGF-beta.

In some embodiments, the antigen-binding moiety (carrier moiety) includes an antibody or fragment thereof known in the art that binds to PD-1 and disrupts the interaction between the PD-1 and its ligand (PD-L1) to stimulate an anti-tumor immune response. In some embodiments, the antibody or antigen-binding portion thereof binds specifically to PD-1. For example, antibodies that target PD-1 and which can find use in the present invention include, but are not limited to, nivolumab (BMS-936558, Bristol-Myers Squibb), pembrolizumab (lambrolizumab, MK03475 or MK-3475, Merck), humanized anti-PD-1 antibody JS001 (ShangHai JunShi), monoclonal anti-PD-1 antibody TSR-042 (Tesaro, Inc.), pidilizumab (anti-PD-1 mAb CT-011, Medivation), anti-PD-1 monoclonal Antibody BGB-A317 (BeiGene), and/or anti-PD-1 antibody SHR-1210 (ShangHai HengRui), human monoclonal antibody REGN2810 (Regeneron), human monoclonal antibody MDX-1106 (Bristol-Myers Squibb), and/or humanized anti-PD-1 IgG4 antibody PDR001 (Novartis). In some embodiments, the PD-1 antibody is from clone: RMP1-14 (rat IgG)-BioXcell cat #BP0146. Other suitable anti-PD-1 antibodies include those disclosed in U.S. Pat. No. 8,008,449. In some embodiments, the antibody or antigen-binding portion thereof binds specifically to PD-L1 and inhibits its interaction with PD-1, thereby increasing immune activity. Any antibodies known in the art which bind to PD-L1 and disrupt the interaction between the PD-1 and PD-L1, and stimulates an anti-tumor immune response, are suitable for use in combination treatment methods disclosed herein. As an example, antibodies that target PD-L1 include BMS-936559 (Bristol-Myers Squibb) and MPDL3280A (Genetech; currently in human trials). Other suitable antibodies that target PD-L1 are disclosed in U.S. Pat. No. 7,943,743. It will be understood by one of ordinary skill that any antibody which binds to PD-1 or PD-L1, disrupts the PD-1/PD-L1 interaction, and stimulates an anti-tumor immune response, is suitable for use in the combination treatment methods disclosed herein.

In some embodiments, wherein the carrier is an antibody against human PD-L1 is selected from ASKB1296, avelumab, atezolizumab and durvalumab.

Exemplary antigen-binding moieties include trastuzumab, rituximab, brentuximab, cetuximab, panitumumab, GC33 (or a humanized version thereof), anti-EGFR antibody mAb806 (or a humanized version thereof), anti-FAP-alpha antibody sibrotuzumab (BIBH1), and fragments thereof. In some embodiments, the antigen-binding moiety that has at least 90%, 91%, 92%, 93%, 94%, 95%, 96%, 97%, 98%, or 99% identity to trastuzumab, rituximab, brentuximab, cetuximab, or panitumumab, GC33 (or a humanized version thereof), anti-EGFR antibody mAb806 (or a humanized version thereof), sibrotuzumab (BIBH1), or a fragment thereof. In some embodiments, the antigen-binding moiety has an antibody heavy chain with at least 90%, 91%, 92%, 93%, 94%, 95%, 96%, 97%, 98%, or 99% identity to the antibody heavy chain of trastuzumab, rituximab, brentuximab, cetuximab, panitumumab, GC33 (or a humanized version thereof), anti-EGFR antibody mAb806 (or a humanized version thereof), sibrotuzumab (BIBH1), or a fragment thereof. In some embodiments, the antigen-binding moiety has an antibody light chain with at least 90%, 91%, 92%, 93%, 94%, 95%, 96%, 97%, 98%, or 99% identity to the antibody light chain of trastuzumab, rituximab, brentuximab, cetuximab, panitumumab, GC33 (or a humanized version thereof), anti-EGFR antibody mAb806 (or a humanized version thereof), sibrotuzumab (BIBH1), or a fragment thereof.

In some embodiments, the antigen-binding moiety comprises the six complementarity determining regions (CDRs) of trastuzumab, rituximab, brentuximab, cetuximab, panitumumab, GC33, anti-EGFR antibody mAb806, or sibrotuzumab (BIBH1). A number of CDR delineations are known in the art and are encompassed herein. A person of skill in the art can readily determine a CDR for a given delineation based on the sequence of the heavy or light chain variable region. The “Kabat” Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, J. Mol. Biol. (1987) 196:901-917). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid number of antibodies refers to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat Numbering) | | | | |
| VH-CDR1 | H31-H35 | H26-H35 | H26-H32 | H30-H35 |
| (Chothia Numbering) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the IL-12 chimeric molecule or prodrug of the present disclosure comprises a carrier comprising an antigen-binding moiety; wherein the antigen-binding moiety binds to Trop-2. In some embodiments, the IL-12 prodrug is used to treat patients with solid tumors. In some embodiments, the prodrug is used to treat triple negative breath cancer, urothelial cancer, small-cell lung cancer, pancreatic cancer, hilar cholangiocarcinoma, cervical cancer, and gastric cancer.

In some embodiment, the IL-12 chimeric molecule or prodrug comprises a carrier comprising an antigen-binding moiety; wherein the antigen-binding moiety binds to 5T4. In some embodiments, the IL-12 prodrug is used to treat patients with solid tumor. In some embodiments, the prodrug is used to treat triple negative breath cancer, small-cell lung cancer, non-small cell lung cancer, pancreatic cancer, ovarian cancer, and gastric cancer.

In some embodiments, the IL-12 prodrug comprises a carrier comprising an antigen-binding moiety; wherein an antigen-binding moiety binds to Claudin 18.2. In some embodiments, the IL-12 prodrug is used to treat patients with pancreatic cancer and gastric cancer.

In some embodiments, an IL-12 chimeric molecule or prodrug comprises a carrier comprising an antigen-binding moiety; wherein a antigen-binding moiety binds to EGFR Type Ill. In some embodiment, an IL-12 prodrug is/are used to treat patients with glioblastoma and colon cancer.

In some embodiment, an IL-12 chimeric molecule/prodrugs is/are used in combination with an immune checkpoint blockade, such as a PD-1 antibody or an PD-1 antibody fragment thereof.

### C. Linker Components of the Chimeric Molecules or Prodrugs

The IL-12 agonist polypeptide is fused to the carrier moiety through a cleavable peptide linker, a non-cleavable peptide linker, or without a peptide linker. The masking moiety is fused to the cytokine moiety, to the carrier, or to another mask through a cleavable or non-cleavable linker, or without a peptide linker.

The non-cleavable peptide linker comprises an amino acid sequence selected from GSEPKSS, GGGGS, GGGGSGGGGS, GGGGSGGGGSGGGGS, or GGGGSGGGGSAAGGGGSGGGGS. In an embodiment, a p35 subunit is directly fused to the p40 subunit through a peptide linker, which comprises 5 to 10 amino acids. In an embodiment, a p35 subunit is directly fused to the p40 subunit through a peptide linker, which comprises GGGGS or GGGGSGGGGS.

The cleavable peptide linker comprises an amino acid sequence selected from SEQ ID NOs: 25-45. In an embodiment, a cleavable linker comprises an amino acid sequence of SEQ ID NO: 32.

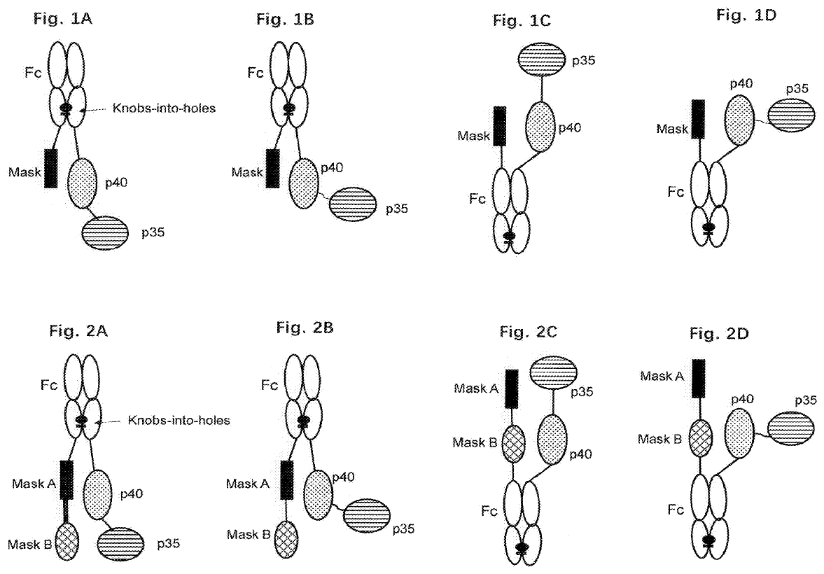

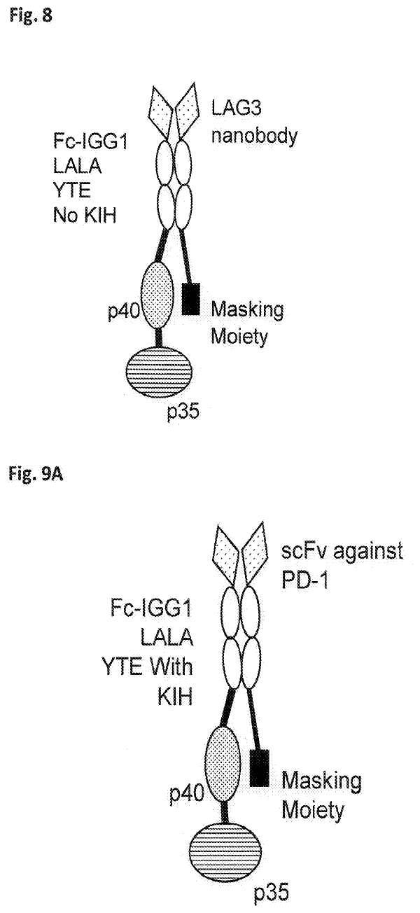

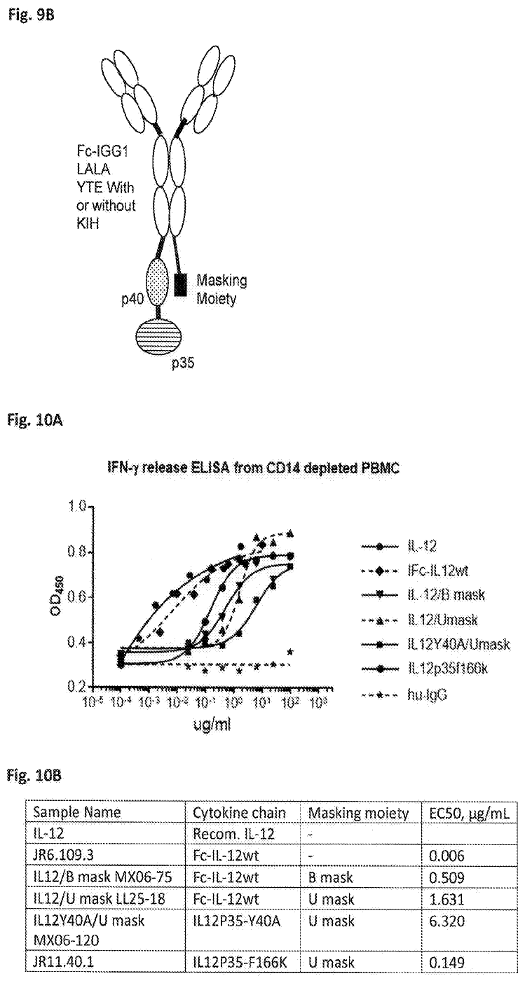

In an embodiment, a chimeric molecule comprises a structure as illustrated in FIG. 1A, FIG. 1B, FIG. 8, FIG. 9A, or FIG. 9B. In an embodiment, a chimeric molecule comprise an IL-12 p40 subunit polypeptide, an IL-12 p35 subunit polypeptide, a masking moiety (MM), a carrier (C), and one or more cleavable and/or non-cleavable peptide linkers; wherein a carried comprises a first polypeptide chain, a second polypeptide chain, and optionally additional polypeptide chain; wherein a masking moiety is linked to the first polypeptide chain of the carrier optionally through a cleavable or non-cleavable peptide linker; wherein a p40 subunit polypeptide chain is directly fused to the second polypeptide chain of the carrier optionally through a cleavable or non-cleavable peptide linker; and wherein a p35 subunit polypeptide is directly fused to the p40 subunit polypeptide through a peptide linker. In an embodiment, a peptide linker comprises 5 to 10 amino acids. In an embodiment, peptide linker comprises GGGGS or GGGGSGGGGS.

D. IL-12 Chimeric Molecule/Prodrugs with an Additional Effector Polypeptide

### 1. CD40DL Agonist Polypeptides

In some embodiments, the IL-12 chimeric molecule or prodrug of the present disclosure further comprises another cytokine or effector moiety. In some embodiments, the second effector moiety comprises one or more copies of the ectodomain of 4-1 BBL, CD40L, OX40L, or GITRL. In some embodiment, a chimeric molecule or prodrug comprises 1, 2, 3, 4, or 6 copies of the ectodomain of CD40L. In some embodiment, a CD40L ectodomain comprises an amino acid sequence of SEQ ID NO: 7 or at least 80%, at least 85%, at least 90%, at least 95%, or at least 99% identical as that of SEQ ID NO: 7.

### 2. NKG2D Ligand Polypeptides

In some embodiments, the IL-12 chimeric molecule or prodrug of the present disclosure further comprises one or more copies of the ectodomain of a NKG2D ligand. In some embodiment, an NKG2D ligand comprises an amino acid sequence of SEQ ID NO: 68 or or at least 80%, at least 85%, at least 90%, at least 95%, or at least 99% identical as that of SEQ ID NO: 68.

### II. Examples of IL-12 Chimeric Molecules or Prodrugs

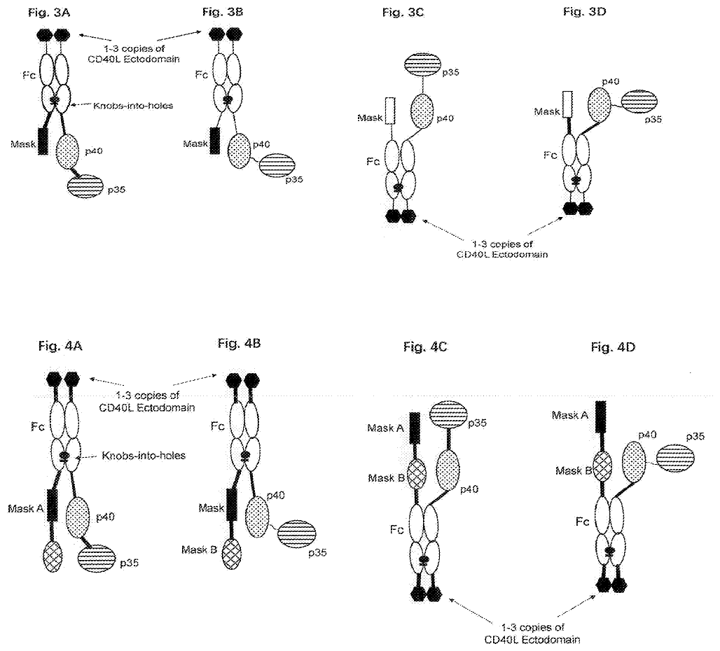

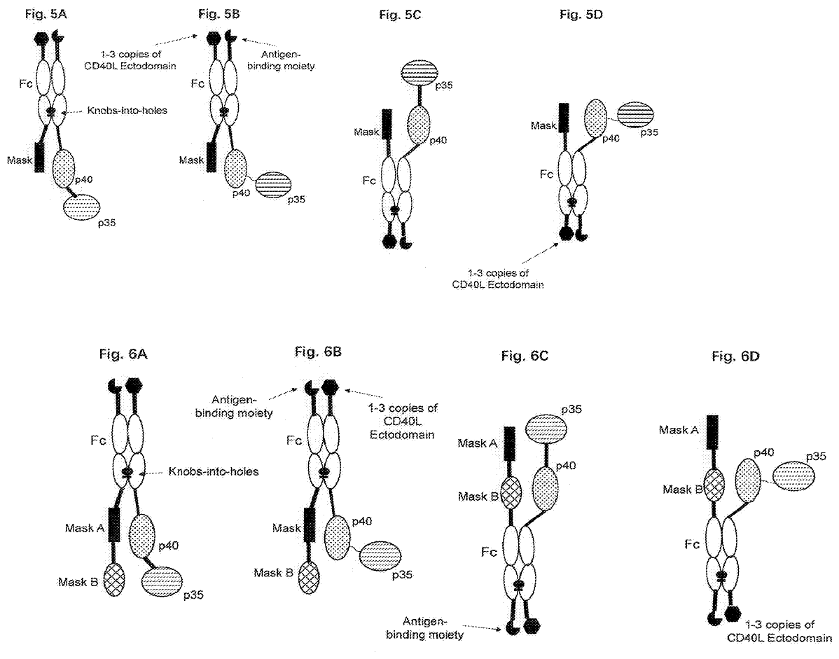

In some embodiments, the IL-12 chimeric molecules or prodrug comprises an antibody or an Fc domain, and an IL-12 agonist polypeptide; wherein the IL-12 agonist polypeptide is fused to the C-terminal of one of the heavy chains of the antibody or Fc polypeptide chain of the Fc domain, optionally through a peptide linker; wherein an IL-12 masking moiety is fused to the C-terminal of the 2^{nd} heavy chain or Fc polypeptide chain. Examples of the chimeric molecules or prodrugs are illustrated in FIGS. 1A, 1B, 1C, 1D, 2A, 2B, 2C, 2D, 3A, 3B, 3C, 3D, 4A, 4B, 4C, 4D, 5A, 5B, 5C, 5D, 6A, 6B, 6C, and 6D. All the chimeric molecules or prodrugs are shown with the cytokine components p35 and p40 and the masking moieties located on the C-termini of the Fc domains. In some embodiments, the cytokine components p35 and p40 and the masking moieties are located on the N-termini of the Fc domains.

By way of example, an IL-12 chimeric molecule comprises a first polypeptide chain and a second polypeptide chain; wherein a first polypeptide chain comprises an amino acid sequence of SEQ ID NO: 80 or at least 90% identical as that of SEQ ID NO: 80, and a second polypeptide chain comprises an amino acid sequence of SEQ ID NO: 81 or at least 90% identical as that of SEQ ID NO: 81.

By way of example, an IL-12 chimeric molecules or prodrugs of Group A comprises three polypeptide chains, wherein the first polypeptide chain comprises an amino acid sequence at least 99% identical to SEQ ID NO: 8, 47, or 49; the second polypeptide chain comprises an amino acid sequence at least 99% identical as on selected from SEQ ID NOs: 13-17, 57-59, and 60; and the third polypeptide chain comprises an amino acid sequence at least 90% identical as that of SEQ ID NO: 6. For each molecule in this group, the IL-12 cytokine moiety and the masking moiety are fused to the C-termini of the Fc domain.

By way of example, an IL-12 chimeric molecules or prodrugs of Group B comprises three polypeptide chains, wherein the first polypeptide chain comprises an amino acid sequence at least 99% identical to SEQ ID NO: 9, 48, or 50; the second polypeptide chain comprises an amino acid sequence at least 99% identical as on selected from SEQ ID NOs: 18-20, 61, and 62; and the third polypeptide chain comprises an amino acid sequence at least 90% identical as that of SEQ ID NO: 6. For each molecule in this group, the IL-12 cytokine moiety and the masking moiety are fused to the N-termini of the Fc domain.

By way of example, an IL-12 chimeric molecules or prodrugs of Group C comprises two polypeptide chains, wherein the first polypeptide chain comprises an amino acid sequence at least 99% identical to SEQ ID NO: 10, 11, 21, 23, 51, 52, 54, or 55; and the second polypeptide chain comprises an amino acid sequence at least 99% identical as on selected from SEQ ID NOs: 13-17, 57-59, and 60. For each molecule in this group, the IL-12 cytokine moiety and the masking moiety are fused to the C-termini of the Fc domain.

By way of example, an IL-12 chimeric molecules or prodrugs of Group D comprises two polypeptide chains, wherein the first polypeptide chain comprises an amino acid sequence at least 99% identical to SEQ ID NO: 12, 22, 24, 53, or 56; the second polypeptide chain comprises an amino acid sequence at least 99% identical as on selected from SEQ ID NOs: 18-20, 61, and 62. For each molecule in this group, the IL-12 cytokine moiety and the masking moiety are fused to the N-termini of the Fc domain.

TABLE 2
Exemplary Chimeric Molecules

| Molecule | | | Third chain if |
|---|---|---|---|
| Group | Cytokine Chain | Mask Chain | any |
| A | SEQ ID NO: 8-Fc-IL-12 | SEQ ID NO: 13 Fc-scFV which | SEQ ID NO: 6 |
| | P40 with knobs mutations | binds to IL-12-VL-VH | P35 subunit of IL- |
| | SEQ ID NO: 47-Fc-IL-12 | Ustekinumab-Briakinnumab | 12 |
| | P40 mutant with knobs | SEQ ID NO: 14 Fc-scFV which | |
| | mutations | binds to IL-12-VL-VH | |
| | SEQ ID NO: 49-Fc-IL-12 | Briakinnumab-Ustekinumab | |
| | mutant P40 with knobs | SEQ ID NO: 15 Fc-scFV which | |
| | mutations | binds to IL-12-VL-VH | |
| | | Briakinnumab-Ustekinumab | |
| | | with cleavable linker | |
| | | SEQ ID NO: 57 Fc-scFV which | |
| | | binds to IL-12-VL-VH | |
| | | Ustekinumab | |
| | | SEQ ID NO: 58 Fc-scFV which | |
| | | binds to IL-12-VL-VH | |
| | | Ustekinumab with cleavable | |
| | | linker | |
| | | SEQ ID NO: 16 CD40L-Fc- | |
| | | scFV which binds to IL-12-VL- | |
| | | VH Ustekinumab-Briakinnumab | |
| | | SEQ ID NO: 17 CD40L-Fc- | |
| | | scFV which binds to IL-12-VL- | |
| | | VH Briakinnumab-Ustekinumab | |
| | | with cleavable linker | |
| | | SEQ ID NO: 59 CD40L-Fc- | |
| | | scFV which binds to IL-12-VL- | |
| | | VH Ustekinumab | |
| | | SEQ ID NO: 60 CD40L-Fc- | |
| | | scFV which binds to IL-12-VL- | |
| | | VH Briakinnumab-Ustekinumab | |
| | | with cleavable linker | |
| B | SEQ ID NO: 9-IL-12 P40-Fc | SEQ ID NO: 18 scFV which | SEQ ID NO: 6 |
| | with knobs mutations | binds to IL-12-VL-VH | P35 subunit of IL- |
| | SEQ ID NO: 48-IL-12 P40 | Ustekinumab-Briakinnumab- | 12 |
| | mutant-Fc with knobs | Fc-CD40L | |
| | mutations | SEQ ID NO: 19 scFV which | |
| | SEQ ID NO: 50-IL-12 P40 | binds to IL-12-VL-VH | |
| | mutant-Fc with knobs | Ustekinumab-Briakinnumab- | |
| | mutations | Fc-CD40L, with a cleavable | |
| | | linker | |
| | | SEQ ID NO: 20 scFV which | |
| | | binds to IL-12-VL-VH | |
| | | Briakinnumab-Ustekinumab- | |
| | | Fc-CD40L | |
| | | SEQ ID NO: 61 scFV which | |
| | | binds to IL-12-VL-VH | |
| | | Ustekinumab-Fc-CD40L | |
| | | SEQ ID NO: 62 scFV which | |
| | | binds to IL-12-VL-VH | |
| | | Ustekinumab-Fc-CD40L, | |
| | | with a cleavable linker | |
| C | SEQ ID NO: 10-Fc-IL-12 | SEQ ID NO: 13 Fc-scFV which | |
| | (P40-P35) with knobs | binds to IL-12-VL-VH | |
| | mutations | Ustekinumab-Briakinnumab | |
| | SEQ ID NO: 51-Fc-IL-12 | SEQ ID NO: 14 Fc-scFV which | |
| | (P40-P35) mutant with knobs | binds to IL-12-VL-VH | |
| | mutations | Briakinnumab-Ustekinumab | |
| | SEQ ID NO: 54-Fc-IL-12 | SEQ ID NO: 15 Fc-scFV which | |
| | (P40-P35) mutant with knobs | binds to IL-12-VL-VH | |
| | mutations | Briakinnumab-Ustekinumab | |
| | SEQ ID NO: 11 CD40L-Fc- | with cleavable linker | |
| | IL-12 (P40-P35) with knobs | SEQ ID NO: 57 Fc-scFV which | |
| | mutations | binds to IL-12-VL-VH | |
| | SEQ ID NO: 21-SIRPα ECD- | Ustekinumab | |
| | Fc-IL-12 (P40-P35) with | SEQ ID NO: 58 Fc-scFV which | |
| | knobs mutations | binds to IL-12-VL-VH | |
| | SEQ ID NO: 23-CD47 ECD- | Ustekinumab with cleavable | |
| | Fc-IL-12 (P40-P35) with | linker | |
| | knobs mutations | SEQ ID NO: 16 CD40L-FC- | |
| | SEQ ID NO: 52 CD40L-Fc- | scFV which binds to IL-12-VL- | |
| | IL-12 (P40-P35) mutant with | VH Ustekinumab-Briakinnumab | |
| | knobs mutations | SEQ ID NO: 17 CD40L-Fc- | |
| | SEQ ID NO: 55 CD40L-Fc- | scFV which binds to IL-12-VL- | |
| | IL-12 (P40-P35) mutant with | VH Briakinnumab-Ustekinumab | |
| | knobs mutations | with cleavable linker | |
| | | SEQ ID NO: 59 CD40L-Fc- | |
| | | scFV which binds to IL-12-VL- | |
| | | VH Ustekinumab | |
| | | SEQ ID NO: 60 CD40L-Fc- | |
| | | scFV which binds to IL-12-VL- | |
| | | VH Briakinnumab-Ustekinumab | |
| | | with cleavable linker | |
| D | SEQ ID NO: 12 IL-12 (P40- | SEQ ID NO: 18 scFV which | |
| | P35)-Fc-CD40L with knobs | binds to IL-12-VL-VH | |
| | mutations | Ustekinumab-Briakinnumab- | |
| | SEQ ID NO: 22-IL-12 (P40- | Fc-CD40L | |
| | P35)-Fc-SIRPα ECD with | SEQ ID NO: 19 scFV which | |
| | knobs mutations | binds to IL-12-VL-VH | |
| | SEQ ID NO: 24-IL-12 (P40- | Ustekinumab-Briakinnumab- | |
| | P35)-Fc-CD47 ECD with | Fc-CD40L, with a cleavable | |
| | knobs mutations | linker | |
| | SEQ ID NO: 53 IL-12 (P40- | SEQ ID NO: 20 scFV which | |
| | P35) mutant-Fc-CD40L | binds to IL-12-VL-VH | |
| | with knobs mutations | Briakinnumab-Ustekinumab- | |
| | SEQ ID NO: 56 IL-12 (P40- | Fc-CD40L | |
| | P35) mutant-Fc-CD40L | SEQ ID NO: 61 scFV which | |
| | with knobs mutations | binds to IL-12-VL-VH | |
| | | Ustekinumab-Fc-CD40L | |
| | | SEQ ID NO: 62 scFV which | |
| | | binds to IL-12-VL-VH | |
| | | Ustekinumab-Fc-CD40L, | |
| | | with a cleavable linker | |

By way of example, an IL-12 chimeric molecule or prodrug comprises two polypeptide chains, wherein the first polypeptide chain comprises an amino acid sequence at least 99% identical to SEQ ID NO: 97; the second polypeptide chain comprises an amino acid sequence at least 99% identical as on selected from SEQ ID NOs: 84, 89, 90, 91, 93, and 96. For each molecule in this group, the IL-12 cytokine moiety and the masking moiety are fused to the C-termini of the Fc domain. In addition, each of the IL-12 cytokine moiety comprises one or more mutations in its p35 or p40 subunit, or a deletion of several amino acids in its p40 subunit.

TABLE 3
Exemplary Chimeric Molecules Comprising Mutant p40 Subunit or
Mutant p35 Subunit

| Sample Name | 1^{st} Polypeptide chain | 2^{nd} polypeptide chain |
|---|---|---|
| IL12p35-y40A | SEQ ID NO: 97 | SEQ ID NO: 84 |
| IL12p35-F166K | | SEQ ID NO: 90 |
| IL12p40-delection | | SEQ ID NO: 93 |
| IL-12p40-AA | | SEQ ID NO: 96 |
| IL-12p35-N71Q | | SEQ ID NO: 89 |
| IL-12p35-T43A/S44G | | SEQ ID NO: 91 |
| IL-12 wt U mask | | SEQ ID NO: 81 |

### III. Pharmaceutical Compositions

Pharmaceutical compositions of the prodrugs are prepared by mixing the presently disclosed prodrugs, or antibody fusion molecules or the antibody fusion molecule drug conjugate having the desired degree of purity with one or more optional pharmaceutically acceptable carriers (see Osol, A. Ed. Remington's Pharmaceutical Sciences 16th edition (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC®polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may comprise as—or in addition to—the carrier, excipient or diluent any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be administered by a number of routes. In some embodiment, a formulation is administrated directly in a tumor or tumors.

In some embodiments, an antibody or protein formulation is a lyophilized formulation. In another embodiment, an antibody or protein formulation is an aqueous formulation.

In some embodiments, the pharmaceutical composition is a combination pharmaceutical composition, which comprises an IL-12 chimeric molecule or prodrug of the present invention, a pharmaceutically acceptable excipient, and a second active ingredient selected from a cytokine other than IL-12 or its fusion molecule, an antibody against PD-1, an antibody against PD-L1, an antibody against CTLA-4, an antibody against CD47, a PD-1 antibody-IL-15 fusion molecule, a PD-1-IL-2 fusion molecule, and a PD-1-IL-21 fusion molecule.

In other aspects of this embodiment, a chimeric molecule disclosed herein reduces the size of a tumor by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a chimeric molecule disclosed herein reduces the size of a tumor from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein may comprise a therapeutic compound in an amount sufficient to allow customary administration to an individual. In aspects of this embodiment, a pharmaceutical composition disclosed herein may be, e.g., at least 5 μg, at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg, at least 150 μg, at least 200 μg, at least 250 μg, at least 300 μg, at least 350 μg, at least 400 μg, at least 450 μg, at least 500 μg, at least 550 μg, at least 600 μg, at least 650 μg, at least 700 μg, at least 750 μg, at least 800 μg, at least 850 μg, at least 900 μg, at least 950 μg, at least 1000 μg, at least 1500 μg, at least 2000 μg, at least 25000 μg, at least 3000 μg, at least 3500 μg, at least 4000 μg, at least 4500, at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a chimeric molecule.

In other aspects of this embodiment, a pharmaceutical composition disclosed herein may be, e.g., t least 5 μg, at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg, at least 150 μg, at least 200 μg, at least 250 μg, at least 300 μg, at least 350 μg, at least 400 μg, at least 450 μg, at least 500 μg, at least 550 μg, at least 600 μg, at least 650 μg, at least 700 μg, at least 750 μg, at least 800 μg, at least 850 μg, at least 900 μg, at least 950 μg, at least 1000 μg, at least 1500 μg, at least 2000 μg, at least 25000 μg, at least 3000 μg, at least 3500 μg, at least 4000 μg, at least 4500, at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a chimeric compound.

In yet other aspects of this embodiment, a pharmaceutical composition disclosed herein may be in the range of, e.g., about 5 μg to about 100 μg, about 10 μg to about 100 μg, about 50 μg to about 150 μg, about 100 μg to about 250 μg, about 150 μg to about 350 μg, about 250 μg to about 500 μg, about 350 μg to about 600 μg, about 500 μg to about 750 μg, about 600 μg to about 900 μg, about 750 μg to about 1,000 μg, about 850 μg to about 1,200 μg, or about 1,000 μg to about 1,500 μg.

In still other aspects of this embodiment, a pharmaceutical composition disclosed herein may be in the range of, e.g., about 10 μg to about 250 μg, about 10 μg to about 500 μg, about 10 μg to about 750 μg, about 10 μg to about 1,000 μg, about 10 μg to about 1,500 μg, about 50 μg to about 250 μg, about 50 μg to about 500 μg, about 50 μg to about 750 μg, about 50 μg to about 1,000 μg, about 50 μg to about 1,500 μg, about 100 μg to about 250 μg, about 100 μg to about 500 μg, about 100 μg to about 750 μg, about 100 μg to about 1,000 μg, about 100 μg to about 1,500 μg, about 200 μg to about 500 μg, about 200 μg to about 750 μg, about 200 μg to about 1,000 μg, about 200 μg to about 1,500 μg, about 5 mg to about 1,500 mg, about 5 mg to about 1,000 mg, or about 5 mg to about 250 mg, about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg of a chimeric molecule.

A pharmaceutical composition disclosed herein may comprise a solvent, emulsion or other diluent in an amount sufficient to dissolve a therapeutic compound disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v).

In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a chimeric molecule disclosed herein in a pharmaceutical composition disclosed herein may be of any concentration desired. In an aspect of this embodiment, the final concentration of a chimeric molecule in a pharmaceutical composition may be a therapeutically effective amount.

In other aspects of this embodiment, the final concentration of a chimeric molecule in a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL, at least 500 mg/mL, at least 700 mg/mL, at least 1,000 mg/mL, or at least 1,200 mg/mL. In other aspects of this embodiment, the concentration of a therapeutic compound disclosed herein in the solution may be, e.g., at most 1,000 mg/mL, at most 1,100 mg/mL, at most 1,200 mg/mL, at most 1,300 mg/mL, at most 1,400 mg/mL, at most 1,500 mg/mL, at most 2,000 mg/mL, at most 2,000 mg/mL, or at most 3,000 mg/mL.

In other aspects of this embodiment, the final concentration of a chimeric molecule in a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual suffering from cancer. As used herein, the term “treating,” refers to reducing or eliminating in an individual a clinical symptom of cancer; or delaying or preventing in an individual the onset of a clinical symptom of cancer. For example, the term “treating” can mean reducing a symptom of a condition characterized by a cancer, including, but not limited to, tumor size, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%.

The actual symptoms associated with cancer are well known and can be determined by a person of ordinary skill in the art by taking into account factors, including, without limitation, the location of the cancer, the cause of the cancer, the severity of the cancer, and/or the tissue or organ affected by the cancer. Those of skill in the art will know the appropriate symptoms or indicators associated with a specific type of cancer and will know how to determine if an individual is a candidate for treatment as disclosed herein.

In aspects of this embodiment, a therapeutically effective amount of a chimeric molecule disclosed herein reduces a symptom associated with cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a chimeric molecule disclosed herein reduces a symptom associated with cancer by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%.

In yet other aspects of this embodiment, a therapeutically effective amount of a chimeric molecule disclosed herein reduces a symptom associated with cancer by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a chimeric molecule disclosed herein generally is in the range of about 0.001 mg/kg/day to about 100 mg/kg/day. In aspects of this embodiment, an effective amount of a chimeric molecule disclosed herein may be, e.g., at least 0.001 mg/kg/day, at least 0.01 mg/kg/day, at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day.

In other aspects of this embodiment, an effective amount of a chimeric molecule disclosed herein may be in the range of, e.g., about 0.001 mg/kg/day to about 10 mg/kg/day, about 0.001 mg/kg/day to about 15 mg/kg/day, about 0.001 mg/kg/day to about 20 mg/kg/day, about 0.001 mg/kg/day to about 25 mg/kg/day, about 0.001 mg/kg/day to about 30 mg/kg/day, about 0.001 mg/kg/day to about 35 mg/kg/day, about 0.001 mg/kg/day to about 40 mg/kg/day, about 0.001 mg/kg/day to about 45 mg/kg/day, about 0.001 mg/kg/day to about 50 mg/kg/day, about 0.001 mg/kg/day to about 75 mg/kg/day, or about 0.001 mg/kg/day to about 100 mg/kg/day.

In yet other aspects of this embodiment, an effective amount of a chimeric molecule disclosed herein may be in the range of, e.g., about 0.01 mg/kg/day to about 10 mg/kg/day, about 0.01 mg/kg/day to about 15 mg/kg/day, about 0.01 mg/kg/day to about 20 mg/kg/day, about 0.01 mg/kg/day to about 25 mg/kg/day, about 0.01 mg/kg/day to about 30 mg/kg/day, about 0.01 mg/kg/day to about 35 mg/kg/day, about 0.01 mg/kg/day to about 40 mg/kg/day, about 0.01 mg/kg/day to about 45 mg/kg/day, about 0.01 mg/kg/day to about 50 mg/kg/day, about 0.01 mg/kg/day to about 75 mg/kg/day, or about 0.01 mg/kg/day to about 100 mg/kg/day.

In still other aspects of this embodiment, an effective amount of a chimeric molecule disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day.

In other aspects of this embodiment, an effective amount of a chimeric molecule disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day.

In yet other aspects of this embodiment, an effective amount of a chimeric molecule disclosed herein may be in the range of, e.g., about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day.

In liquid and semi-solid formulations, a concentration of a chimeric molecule disclosed herein typically may be between about 50 mg/mL to about 1,000 mg/mL. In aspects of this embodiment, a therapeutically effective amount of a chimeric molecule disclosed herein may be from, e.g., about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

Dosing can be single dosage or cumulative (serial dosing), and can be readily determined by one skilled in the art. For instance, treatment of a cancer may comprise a one-time administration of an effective dose of a pharmaceutical composition disclosed herein. Alternatively, treatment of a cancer may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, a chimeric molecule disclosed herein is capable of reducing the number of cancer cells or tumor size in an individual suffering from a cancer by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a chimeric molecule is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a chimeric molecule has half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a chimeric molecule is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a chimeric molecule disclosed herein reduces or maintains a cancer cell population and/or tumor cell size in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a chimeric molecule disclosed herein reduces or maintains a cancer cell population and/or tumor cell size in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a chimeric molecule disclosed herein reduces or maintains a cancer cell population and/or tumor cell size in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

Typically, any individual who is a candidate for treatment is a candidate with some form of cancer, whether the cancer is benign or malignant, a tumor, solid or otherwise, a cancer call not located in a tumor or some other form of cancer. Among the most common types of cancer include, but are not limited to, bladder cancer, breast cancer, colon and rectal cancer, endometrial cancer, kidney cancer, renal cancer, leukemia, lung cancer, melanoma, non-Hodgkins lymphoma, pancreatic cancer, prostate cancer, stomach cancer and thyroid cancer. Pre-operative evaluation typically includes routine history and physical examination in addition to thorough informed consent disclosing all relevant risks and benefits of the procedure.

In still other aspects of this embodiment, a pharmaceutical composition disclosed herein may be in the range of, e.g., about 10 μg to about 250 μg, about 10 μg to about 500 μg, about 10 μg to about 750 μg, about 10 μg to about 1,000 μg, about 10 μg to about 1,500 μg, about 50 μg to about 250 μg, about 50 μg to about 500 μg, about 50 μg to about 750 μg, about 50 μg to about 1,000 μg, about 50 μg to about 1,500 μg, about 100 μg to about 250 μg, about 100 μg to about 500 μg, about 100 μg to about 750 μg, about 100 μg to about 1,000 μg, about 100 μg to about 1,500 μg, about 200 μg to about 500 μg, about 200 μg to about 750 μg, about 200 μg to about 1,000 μg, about 200 μg to about 1,500 μg, about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg of a chimeric molecule.

### IV. Methods of Treatment

The presently disclosed chimeric molecules or prodrugs can be used to treat a disease, depending on the antigen bound by the antigen-binding domain. In some embodiments, the chimeric molecule or prodrugs disclosed herein are used to treat cancer. In some embodiments, the chimeric molecule or prodrugs are used to treat an infection, for example when the drug molecule is an antibacterial agent or an antiviral agent.

In some embodiments, a method of treating a disease (such as cancer, a viral infection, or a bacterial infection) in a subject comprises administering to the subject an effective amount of the presently disclosed chimeric molecule or prodrugs.

In some embodiments, the cancer is a solid cancer. In some embodiments, the cancer is a blood cancer. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer, cholangiocarcinoma, breast cancer, and ovarian cancer.

In some embodiments, the presently disclosed chimeric molecule or prodrugs are used to treat a bacterial infection such as sepsis. In some embodiments, the bacteria causing the bacterial infection are drug-resistant bacteria. In some embodiments, the antigen-binding moiety binds to a bacterial antigen.

In some embodiments, the chimeric molecule is used to treat a viral infection. In some embodiments, the virus causing the viral infection is hepatitis C (HCV), hepatitis B (HBV), human immunodeficiency virus (HIV), a human papilloma virus (HPV). In some embodiments, the antigen-binding moiety binds to a viral antigen.

Generally, dosages and routes of administration of the present pharmaceutical compositions are determined according to the size and condition of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally. In some embodiments, the composition is administered to a subject intravenously.

In some embodiments, the prodrug is administered to a subject in need a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the antibody fusion molecule conjugated to the drug is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 2, 3, 4, 5, 7, 10, 15, or 20 or more doses).

In some embodiments, the IL-12 prodrug is administered to a subject in combination with a second pharmaceutical composition, wherein the second pharmaceutical composition comprises an active ingredient selected from a cytokine other than IL-12 or its fusion molecule, an antibody against PD-1, an antibody against PD-L1, an antibody against CTLA-4, an antibody against CD47, a PD-1 antibody-IL-2 fusion molecule, a PD-1-IL-7 fusion molecule, a PD-1 antibody-IL-15 fusion molecule, and a PD-1-IL-21 fusion molecule.

### V. Methods of Making the Chimeric Molecules or Prodrugs

The presently disclosed chimeric molecules or prodrugs can be produced using recombinant DNA methods. Nucleic acid molecules encoding the polypeptide or the fusion polypeptide of a chimeric molecule or a prodrug can be isolated and inserted into one or more vectors for further cloning and/or expression in a host cell. Such nucleic acid molecules may be readily isolated and sequenced using conventional methods. Suitable host cells for cloning or expression of fusion polypeptide vectors include prokaryotic cells or eukaryotic cells. Exemplary host cells include Chinese Hamster Ovary (CHO) cells or human embryonic kidney cells (e.g., HEK293).

Expression host cells express the chimeric molecule. After an expression period, the host cells can by lysed and the chimeric molecule or the prodrug can be purified. Exemplary purification methods include liquid chromatography, such as ion exchange chromatography, affinity chromatography (such as Protein A affinity chromatography), or size exclusion chromatography.

### EXEMPLARY EMBODIMENTS

Further particular embodiments of the present disclosure are described as follows. These embodiments are intended to illustrate the compositions and methods described in the present disclosure and are not intended to limit the scope of the present disclosure.

1. An isolated mutant interleukin-12 (IL-12) polypeptide comprising a p40 subunit polypeptide and a mutant p35 subunit polypeptide, wherein a mutant p35 polypeptide comprises the amino acid sequence of SEQ ID NO: 6 with up to three amino acid substitutions, wherein one of the mutations is at position 40.

2. An isolated mutant interleukin-12 (IL-12) polypeptide comprising a mutant p35 subunit polypeptide, wherein a mutant p35 subunit polypeptide comprises the amino acid sequence of SEQ ID NO: 6 with up to seven amino acid substitutions, wherein one of the mutations is selected from K128A, F166K, T36A, L37E, E38A, F39A, E38A, F39A, Y40A, P41G, T43A, S44G, E45A, E46A, D48A, H49A, E50A, E79A, T80A, F82A, and N71Q.

3. The mutant interleukin-12 polypeptide of any one of embodiments 1-2, further comprising a mutant p40 subunit polypeptide.

4. The mutant interleukin-12 polypeptide of any one of embodiments 1-3, wherein a mutant p40 subunit comprises Lys260Ala and Arg261Ala (numbering according to SEQ ID NO: 5).

5. The mutant interleukin-12 polypeptide of any one of embodiments 1-3, wherein a p40 subunit comprises an amino acid sequence selected from SEQ ID NOs: 5, 46, 63, and 98 or an amino acid sequence at least 95% identical or at least 99% identical as one selected from SEQ ID NOs: 5, 46, 63, and 98.

6. A chimeric molecule, which comprises the mutant interleukin-12 polypeptide of any one of embodiments 1 to 5 and a carrier, wherein a mutant IL-12 polypeptide is operationally linked to a carrier, wherein a carrier is selected from a PEG molecule, an albumin molecule, an albumin fragment, an IgG Fc, and an antigen binding molecule.

7. Chimeric molecule of embodiment 6, wherein a carrier is an antigen binding molecule, and wherein said antigen binding molecule is an antibody or an antibody fragment.

8. Chimeric molecule of embodiment 6, wherein a carrier is an antigen binding molecule, and wherein said antigen binding molecule is a bispecific antibody.

9. Chimeric molecule of any of embodiments 7 and 8, wherein said antigen is expressed on an immune cell.

10. Chimeric molecule of any of embodiments 7 and 8, wherein said antigen is expressed on a tumor cell or a cell in a tumor microenvironment.

11. Chimeric molecule of any of embodiments 7 and 8, wherein said antigen is selected from the group of PD-L1, PD-I, LAG-3, TIM-3, TIGIT, CD-8, CD16A, NKG2A, NKG2D, NKp46, SIRPalpha, CD47, CD38, Fibroblast Activation Protein alpha (FAPalpha), CEA, BCMA, CD20, Trop-2, HER2, 5T4, the Melanoma-associated Chondroitin Sulfate Proteoglycan (MCSP), PSMA, EGFR, and Claudin 18.2.

12. Chimeric molecule of any of embodiments 7-11, wherein said chimeric molecule further comprises a masking moiety (MM); wherein said masking moiety binds to p40 subunit and/or p35 subunit and inhibits a biological activity of IL-12.

13. Chimeric molecule of any of embodiments 7-11, wherein said chimeric molecule further comprises a masking moiety (MM); wherein said masking moiety comprises an amino acid sequence selected from SEQ ID NOs: 1, 2, 3, and 4, or an amino acid sequence at least 90%, at least 95%, or at least 99% identical as SEQ ID NO: 1, 2, 3, or 4.

14. A chimeric molecules which comprises an IL-12 p40 subunit (p40), an IL-12 p35 subunit (p35), a masking moiety (MM), and a carrier (C); wherein a p40 subunit comprises a mutation, mutations, or a deletion of one or more amino acids; and wherein said masking moiety comprises an amino acid sequence selected from SEQ ID NOs: 1, 2, 3, and 4, or an amino acid sequence at least 90%, at least 95%, or at least 99% identical as SEQ ID NO: 1, 2, 3, or 4.

15. Chimeric molecule of embodiment 14, wherein said p40 subunit comprises an amino wherein a p40 subunit comprises an amino acid sequence selected from SEQ ID NOs: 5, 46, 63, and 98 or an amino acid sequence at least 95% identical or at least 99% identical as one selected from SEQ ID NOs: 5, 46, 63, and 98

16. A chimeric molecule comprises an IL-12 p40 subunit (p40), an IL-12 p35 subunit (p35), a masking moiety (MM), and a carrier (C); wherein said masking moiety (MM) inhibits the binding of the cytokine moiety with the IL-12 receptor β1 subunit; and wherein said p35 subunit comprises mutation or mutations which reduce the binding affinity of the cytokine moiety with the IL-12 receptor β2 subunit.

17. A chimeric molecule comprises an IL-12 p40 subunit (p40), an IL-12 p35 subunit (p35), a masking moiety (MM), and a carrier (C); wherein said masking moiety (MM) inhibits the binding of p40 subunit with the IL-12 receptor β1 subunit; and wherein said p35 subunit comprises mutation or mutations which reduce the binding affinity of the cytokine moiety with the IL-12 receptor β2 subunit.

18. Chimeric molecule of embodiment 16 or 17, wherein a p35 subunit comprises a mutation at Tyr40 (numbering according to SEQ ID NO: 6).

19. Chimeric molecule of embodiment 16 or 17, wherein a p35 subunit comprises the mutation Tyr40Ala (numbering according to SEQ ID NO: 6).

20. Chimeric molecule of any of the embodiments 16-19, wherein said masking moiety comprises an amino acid sequence selected from SEQ ID NOs: 1, 2, 3, and 4, or an amino acid sequence at least 90%, at least 95%, or at least 99% identical as SEQ ID NO: 1, 2, 3, or 4.

21. A chimeric molecule which is activatable at the site of a tumor, in the tumor microenvironment, or near a tumor; wherein a chimeric molecule comprises an IL-12 p40 subunit (p40), an IL-12 p35 subunit (p35), a masking moiety (MM), and a carrier (C); wherein said carrier moiety comprises an antigen-binding moiety, which binds to an antigen expressed on an immune cell; wherein said immune cell is infiltered into a tumor or in the tumor microenvironment or locates near a tumor; and wherein said chimeric molecule has higher activity stimulating an immune cell expressing a targeted antigen compared to the ones without expressing a antigen.

22. Chimeric molecule of embodiment 21, which does not comprises any cleavable peptide linker; or which comprise a cleavable peptide linker, but the activation is not dependent on the cleavage of the cleavable linker.

23. A chimeric molecule, wherein: (a) it comprises IL-12 cytokine moiety, a masking moiety, and a carrier which comprises an antigen-binding moiety which binds to antigen expressed on an immune cell; (b) does not comprises any cleavable peptide linker; and (c) is able to activate an immune cell which expresses an antigen targeted by the carrier.

24. Chimeric molecule of any of the embodiments 21-23, wherein said carrier moiety comprises an antigen-binding moiety, which binds to an antigen selected from PD-1, TIM-3, TIGIT, LAG-3, CD16A, NKG2A, NKG2D, KIR, SIRPalpha, and NKp46.

25. Chimeric molecule of any of embodiments 21-24, wherein said carrier comprises an antigen-binding moiety which binds to PD-1; wherein an antigen-binding moiety comprises the same heavy chain variable domain (VH) and the same light chain variable domain (VL) as that of pembrolizumab or nivolumab.

26. Chimeric molecule of embodiment 25, wherein said VH domain comprises an amino acid sequence of SEQ ID NO: 101 or at least 90% identical as that of SEQ ID NO: 101 and said VL domain comprises an amino acid sequence of SEQ ID NO: 102 or at least 90% identical as that of SEQ ID NO: 102.

27. Chimeric molecule of any of embodiments 21-24, wherein said carrier comprises an antigen-binding moiety which binds to LAG-3; wherein said LAG-3-binding moiety comprises an amino acid sequence selected from SEQ ID NOs: 69-77, or at least 90% identical to one selected from SEQ ID NOs: 69-77.

28. A chimeric molecule comprises an IL-12 p40 subunit (p40), an IL-12 p35 subunit (p35), a masking moiety (MM), a carrier (C), and a cleavable peptide linker which is cleavable by a protease preferentially expressed at the site of a tumor, in the tumor microenvironment, or near a tumor; wherein said carrier comprises an Fc domain of an IgG; wherein said masking moiety is linked directly or indirectly to the carrier through a non-cleavable peptide linker; and wherein said p40 subunit of IL-12 is linked to the carried directly or indirectly through a cleavable peptide linker.

29. A chimeric molecule comprises an IL-12 p40 subunit (p40), an IL-12 p35 subunit (p35), a masking moiety (MM), a carrier (C), and a cleavable peptide linker which is cleavable by a protease preferentially expressed at the site of a tumor, in the tumor microenvironment, or near a tumor; wherein said carrier comprises an antigen-binding moiety; wherein said masking moiety is linked directly or indirectly to the carrier through a cleavable peptide linker; and wherein said p40 subunit of IL-12 is linked to the carried directly or indirectly optionally through a non-cleavable peptide linker.

30. Chimeric molecule of embodiment 6, which further comprises a cleavable peptide linker which is cleavable by a protease preferentially expressed at the site of a tumor, in the tumor microenvironment, or near a tumor; wherein said carrier comprises an Fc domain of an IgG; wherein said masking moiety is linked directly or indirectly to the carrier through a non-cleavable peptide linker; and wherein said p40 subunit of IL-12 is linked to the carried directly or indirectly through a cleavable peptide linker.

31. Chimeric molecule of embodiment 6, which further comprises a cleavable peptide linker which is cleavable by a protease preferentially expressed at the site of a tumor, in the tumor microenvironment, or near a tumor; wherein said carrier comprises an antigen-binding moiety; wherein said masking moiety is linked directly or indirectly to the carrier through a cleavable peptide linker; and wherein said p40 subunit of IL-12 is linked to the carried directly or indirectly optionally through a non-cleavable peptide linker.

32. A chimeric molecule, which comprises an IL-12 p40 subunit (p40), an IL-12 p35 subunit (p35), a masking moiety (MM), and a carrier (C); wherein said masking moiety is directly fused to the carrier optionally through a cleavable or non-cleavable peptide linker; wherein said p40 subunit is directly fused to the carrier optionally through a cleavable or non-cleavable peptide linker; and wherein said p35 subunit is fused to the p40 subunit through a peptide linker of 3 to 10 amino acids in length, optionally comprising GGS, GGGGS or GGGGSGGGGS.

33. Chimeric molecule of any of embodiments 6-31, wherein said masking moiety is directly fused to the carrier optionally through a cleavable or non-cleavable peptide linker; wherein said p40 subunit is directly fused to the carrier optionally through a cleavable or non-cleavable peptide linker; and wherein said p35 subunit is fused to the p40 subunit through a peptide linker of of 5 to 10 amino acids in length, optionally comprising GGGGS or GGGGSGGGGS.

34. Chimeric molecule of embodiment 32, wherein said carrier moiety comprises an antigen-binding moiety, which binds an antigen selected from PD-1, TIM-3, TIGIT, LAG-3, CD16A, NKG2A, NKG2D, KIR, SIRPalpha, and NKp46.

35. Chimeric molecule of embodiment 32, wherein said carrier comprises an antigen-binding moiety which binds to PD-1; wherein said antigen-binding moiety comprises the same heavy chain variable domain (VH) and the same light chain variable domain (VL) as that of pembrolizumab or nivolumab.

36. Chimeric molecule of embodiment 35, wherein said VH domain comprises an amino acid sequence of SEQ ID NO: 101 or at least 90% identical as that of SEQ ID NO: 101 and said VL domain comprises an amino acid sequence of SEQ ID NO: 102 or at least 90% identical as that of SEQ ID NO: 102.

37. Chimeric molecule of embodiment 32, wherein said carrier comprises an antigen-binding moiety which binds to LAG-3; wherein said LAG-3-binding moiety comprises an amino acid sequence selected from SEQ ID NOs: 69-77, or at least 90% identical to one selected from SEQ ID NOs: 69-77.

38. Chimeric molecule of any of embodiments 32-37, wherein said masking moiety amino acid sequence selected from SEQ ID NOs: 1, 2, 3, and 4, or an amino acid sequence at least 90%, at least 95%, or at least 99% identical as SEQ ID NO: 1, 2, 3, or 4.

39. Chimeric molecule of any of embodiments 32, 34-38, wherein a p40 subunit comprises an amino acid sequence SEQ ID NOs: 5, 46, 63, and 98 or an amino acid sequence at least 95% identical or at least 99% identical as one selected from SEQ ID NOs: 5, 46, 63, and 98.

40. Chimeric molecule of any of embodiments 32, 34-39, wherein a p35 subunit comprises a mutation at Y40 (numbering according to SEQ ID NO: 6).

41. Chimeric molecule of any of embodiments 32, 34-39, wherein said p35 subunit comprises mutation Y40A (numbering according to SEQ ID NO: 6).

42. Chimeric molecule of any of embodiments 32-41, wherein said carrier comprises a first Fc polypeptide and a second Fc polypeptide; and wherein said masking moiety is fused to the N-terminus of the first Fc polypeptide, and a p40 subunit is fused to the N-terminus of the second Fc polypeptide.

43. Chimeric molecule of any of embodiments 32-41, wherein said carrier comprises a first Fc polypeptide and a second Fc polypeptide; and wherein said masking moiety is fused to the C-terminus of the first Fc polypeptide, and a p40 subunit is fused to the C-terminus of the second Fc polypeptide.

44. Chimeric molecule of any of embodiments 32-41, wherein said carrier is an antibody comprises a first heavy chain polypeptide and a second heavy chain polypeptide; and wherein said masking moiety is fused to the N-terminus of the first heavy chain polypeptide, and a p40 subunit is fused to the N-terminus of the second heavy chain polypeptide.

45. Chimeric molecule of any of embodiments 32-41, wherein said carrier is an antibody comprises a first heavy chain polypeptide and a second heavy chain polypeptide; and wherein said masking moiety is fused to the C-terminus of the first heavy chain polypeptide, and a p40 subunit is fused to the C-terminus of the second heavy chain polypeptide.

46. Chimeric molecule of any of embodiments 42-45, wherein said Fc polypeptides or the heavy chain polypeptides comprise knobs-into-holes mutations.

47. Chimeric molecule of any of embodiments 42-45, which has higher main peak purity by SEC-HPLC compared to the chimeric molecules with the same structure except that the linkers between p40 and p35 subunits comprises a peptide linker with amino acid sequence GGGGSGGGGSGGGGS.

48. Chimeric molecule of any one of embodiments 12-22, 24-47, which comprises a cleavable peptide linker, which is cleavable by a protease or proteases found at a tumor site or its surrounding environment.

49. Chimeric molecule of embodiment 48, wherein a cleavable peptide linker comprises a substrate of uPA, MMP2 and/or MMP9.

50. Chimeric molecule of embodiment 48, wherein a cleavable peptide linker comprises substrates of both uPA and MMP2, both uPA and MMP9, or uPA, MMP2 and MMP9.

51. Chimeric molecule of embodiment 48, wherein a cleavable peptide linker contains an enzyme substrate amino acid sequence selected from LSGRSDNH, ISSGLLSS, and GPLGVR.

52. Chimeric molecule of embodiment 48, wherein a cleavable peptide linker comprises an amino acid sequence selected from SEQ ID NOs: 25-45.

53. A polynucleotide or polynucleotides which encodes the mutant IL-12 of any one of embodiments 1-5.

56. A polynucleotide or polynucleotides which encode the chimeric molecule of any one of embodiments 6-52.

54. An expression vector or vectors comprising the polynucleotide or polynucleotides of embodiment 52 or 53.

55. A host cell transfected with the vector of embodiment 54.

56. Host cell of embodiment 55, wherein a host cell has the gene or genes encoding uPA, MMP-2 and/or MMP-9 knocked out.

57. A method of producing a mutant IL-12 of any one of embodiments 1-5, a chimeric molecule of any of embodiments 6-52, comprising culturing the host cell of embodiment 55 or 56.

58. A pharmaceutical composition comprising as active ingredient the mutant IL-12 of any one embodiments 1-5 or the chimeric molecule of any one of embodiments 6-52.

59. A pharmaceutical composition comprising as active ingredient the chimeric molecule of any one of embodiments 6-52.

60. A method of treating breast, lung, pancreatic, esophageal, medullary thyroid, ovarian, uterine, prostatic, testicular, colon, rectal or stomach cancer, or infectious disease in a human subject in need thereof, comprising administering to the human subject a pharmaceutical composition of embodiment 58 or 59.

In order that this invention may be better understood, the following examples are set forth. These examples are for purposes of illustration only and are not to be construed as limiting the scope of the invention in any manner.

### EXAMPLES

### Example 1. Transient Transfection of ExpiCHO Cells

Expression plasmids were co-transfected into 6×10⁶ cell/ml freestyle ExpiCHO cells at 1 μg/ml using ExpiFectamine CHO Transfection kit (Gibco). Various ratios of the expression plasmids were tested in order to achieve higher titer and/or better purity. The cell cultures were harvested 7 days after transfection by centrifuging at 9,000 rpm for 45 min followed by 0.22 μM filtration.

### Example 2. Purification of Chimeric Molecules and SEC-HPLC Purity Analysis

The purifications of the proteins of the IL-12 chimeric molecules were carried out using Protein A affinity chromatography. Additional purification steps were carried out using additional chromatography and filtration steps. For example, chromatography steps with resins such as Capto MMC ImpRes, Capto Adhere, Capto SP, and/or Capto Q were used for further purification of the chimeric molecules.

B mask MX06-75 is a chimeric molecule comprising Fc as the carrier, which further comprises a scFv derived from Briakinnumab as the masking moiety. It was purified using chromatography steps with Capto Adhere and Capto MMC resins after the Protein A affinity purification. Umask LL25-18 is a chimeric molecule comprising Fc as the carrier, which further comprises a scFv derived from Ustekinumab as the masking moiety. It was also purified using chromatography steps with Capto Adhere and Capto MMC resins after the Protein A affinity purification. The purities of the samples were tested by SEC-HPLC. The sequence information of the molecules is listed in Table 4.

TABLE 4
Sequence Information of Chimeric Molecules Comprising Fc as Their Carriers

| Molecule | Chain 1 | | Chain 2 | | ratio | Description |
|---|---|---|---|---|---|---|
| B mask | CX5.43.1 | SEQ ID | CX10.64.2 | SEQ ID | 1:2 | IL-12/B mask |
| MX06-75 | | NO: 81 | | NO: 80 | | non-cleavable |
| U mask | | | CX5.47.4 | SEQ ID | 1:1 | IL-12/U mask |
| LL25-18 | | | | NO: 97 | | cleavable |

ASKG129A, ASKG129B, and ASKG129C are chimeric molecules which comprise the single domain antibody against LAG-3. They were purified using chromatography steps with Capto MMC Impres and Capto Q Impres resins after the Protein A affinity purification. The sequence information of the molecules is listed in Table 5.

TABLE 5
Sequence Information of Chimeric Molecules Comprising A
Single Domain Antibody Against LAG-3 as Their Carriers

| Molecule Name | | Chain 1 (Cytokine) | | Chain 2 (Mask) | | ratio | Description |
|---|---|---|---|---|---|---|---|
| ASKG129A | LL25-55 | CX14.51.3 | SEQ ID | CX14.51.1 | SEQ ID | 1:1 | WT IL-12 |
| | | | NO: 124 | | NO: 115 | | |
| ASKG129B | LL25-58E2 | CX14.51.4 | SEQ ID | CX14.51.1 | | 1:1 | Mutant p35 |
| | | | NO: 137 | | | | with Y40A |
| ASKG129C | LL25-52 | CX14.51.5 | SEQ ID | CX14.51.1 | | 1:1 | Mutant p40 with |
| | | | NO: 128 | | | | K260A/R261A |

SEC-HPLC was carried out using an Agilent 1100 Series of HPLC system with a TSKgel G3000SWXL column (7.8 mmIDX 30 cm, 5 μm particle size) ordered from Tosoh Bioscience. A sample of up to 100 μl was loaded. The column was run with a buffer containing 200 mM K₃PO₄, 250 mM KCl, pH 6.5. The flow rate was 0.5 ml/min. The column was run at room temperature. The protein elution was monitored both at 220 nm and 280 nm.

Alternatively, SEC-HPLC was also carried out using an Agilent 1100 Series of HPLC system with Phenomenex column #H21-260882 (4.6 mmIDX 30 cm, 3 μm particle size) ordered from Phenomenex. A sample of approximately 3 μg of a chimeric molecule was loaded. The column was run with a buffer containing 200 mM K₃PO₄, 250 mM KCl, pH 6.22. The flow rate was 0.35 ml/min. The column was run at 30° C. The protein elution was monitored at 220 nm.

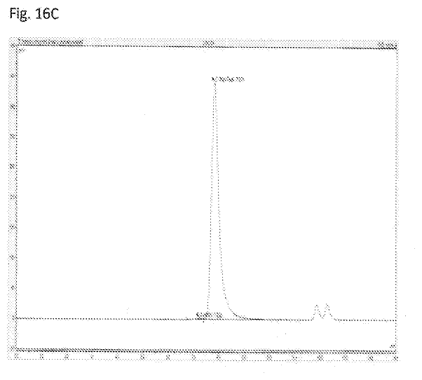

The exemplary SEC-HPLC purities of the purified ASKG129A, ASKG129B and ASKG129C are shown in FIG. 16A (ASKG129A, Lot #LL25-55), 16B (ASKG129B, Lot #LL25-64), and 16C (ASKG129C Lot #LL25-52). The main peak purities were approximately 95% or higher.

### Example 3. SDS-PAGE Analysis

10 μl of the culture supernatants or 4 μg of purified protein samples were mixed with Bolt™ LDS Sample Buffer (Novex) with or without reduce reagents. The samples were heated at 70° C. for 3 min and then loaded to a NuPAGE™ 4-12% BisTris Gel (Invitrogen). The gel was run in NuPAGE™ MOPS SDS Running buffer (Invitrogen) at 200 Volts for 35 min and then stained with Coomassie.

### Example 4. Proteolytic Treatment

One μg of the protease, human MMP-2 (R&D systems), human MMP-9 (R&D systems), mouse MMP-2 (R&D systems), or mouse MMP-9 (R&D systems) was added to 50 μg of the precursor protein, and incubated at 37° C. overnight.

### Example 5. NK92 IL-12 Bioassay

NK92 is an IL-2 dependent Natural Killer (NK) cell derived from peripheral blood of a patient with non-Hodgkin's lymphoma. In the presence of IL-2 NK92 cells will secrete Interferon-g in response to IL-12. Serial dilutions of test articles are performed in 100 mL/well in 96 well tissue culture plates. NK92 cells are added at 40,000 cells/well in 100 mL medium containing 10 ng/mL IL-2. Cultures are incubated at 37° C. for 2 days, and supernatants are assayed for IFN-g by ELISA using commercially available reagents. Data are fitted with a four-parameter logistic (4PL) regression to give the EC₅₀ of the test articles.

### Example 6. HEK-Blue™ IL-12 Bioassay

ASKG129 is assayed for biological function using the HEK-Blue™ IL12 reporter cells line (InvivoGen, Cat. #hkb-il12). IL-12 exerts its biological effects by binding to the IL-12 receptor which is composed of a b1 and b2 chain. Binding of the receptor leads to activation of a signaling pathway involving TyK2 and JAK2 leading to phosphorylation of STAT4 and subsequent production of Interferon-g. HEK-Blue™ IL12 cells have stably introduced genes for the human IL-12 receptor and for the IL-12 signaling pathway. In addition, they express a STAT-4 inducible secreted alkaline phosphatase (SEAP) reportergene. Serial dilutions of test articles are performed in 96 well plates in 50 mL/well assay medium (RPMI 1640, 10% FBS, NEAA, Pyruvate, NEAA, b-mercaptoethanol). Subsequently 30,000 HEK-Blue™ IL12 reporter cell are added in 50 mL/well, and cultures are incubated overnight at 37° C. Twenty microliters of culture supernatant are transferred to a new plate and 180 mL Quanti-Blue™ (Invivogen, Cat. Rep-qb1) reagent is added to each well. After a 1-hour incubation at 37° C., absorbance is measured at 650 nm with a spectrophotometer. Data are fitted with a four-parameter logistic (4PL) regression to give the EC₅₀ of the sample.

### Example 7. Ex Vivo Cytokine Releasing Assay

### Materials and Reagents

The following reagents were used in the cytokine releasing assay to test the ex vivo biological activities of the IL-12 chimeric molecules:
a. PBMCs, which were isolated from buffy coat blood (BiolVT)
b. IFN-g coating antibody (cat #506502, Biolegend), IFN-g detection antibody-biotin (cat #502504, Biolegend), TNF-α coating antibody (cat #502802, Biolegend),
c. TNF-α detection antibody-biotin (cat #502904, Biolegend). Streptavidin-HRP Conjugate (Jackson Immunoresearch Laboratories, cat #016-030-084)
d. Pierce TMB substrates (Thermo Fisher Scientific, cat #34021)
e. ELISA washing buffer (0.05% Tween20 in PBS).

Procedures

### Cell Treatment

Cell treatment was carried out following the procedure listed below:
a. Seeding fresh isolated or frozen PBMC to 96 well tissue culture plate (400k each well).
b. Add designed IL-12 compounds and different concentration to PBMC well (starting con. 100 μg/ml, 1:4 fold dilution. final volume 200 ul in cell culture medium).
c. Culture cells for 72 hrs.
d. pin down plate and take supernatant for ELISA.

### ELISA Analysis

ELISA assay was carried out following the procedure listed below:
a. Coat the wells of the plate with the capture antibody at a concentration of 1 μg/ml in PBS (pH7.2).
b. Block the remaining protein-binding sites in the coated wells by adding 200 μl blocking buffer, 5% BSA/PBST, for at least 1-2 h at room temperature.
c. Add 100 μl of appropriately diluted samples to each well, and Incubate for 90-120 min at room temperature.
d. Add 100 μl of diluted detection antibody (0.5 ug/ml) to each well, and incubate for 1 h at room temperature.
e. Add 100 μl of Streptavidin-HRP Conjugate at 01 μg/ml and incubate for 1 hr. at room temperature.
   Between a-e each step be washed 4× with 200 ul washing buffer.
f. Mixing TMB substrates and dispense 100 μl of the substrate solution per well with a multichannel pipet.
g. Add 100 ul of 1 N H2SO4 to each well when the substrate color development
h. Read the plate with SpectramMax at 450 nm.

### Example 8. pSTAT4 Assay

### Materials and Reagents

The following reagents were used in the pSTAT4 assay to test the ex vivo biological activities of the IL-12 chimeric molecules:
a. RPMI 1640 w/L-glutamine (Life Technologies, 11875-093)
b. 10% Fetal Bovine Serum (Sciencell, #0500)
c. Non-Essential Amino Acids (100×) (Life Technologies, #11140-050)
d. Sodium Pyruvate (100×) (Life Technologies, #11360-070)
e. Penicillin/Streptomycin (Thermo-Fisher Scientific, 15070-063)
f. Beta-mercaptoethanol (1000×) (Life Technologies, #21985-023)
g. Human PBMC (Peripheral blood mononuclear cells) (BIOIVT #RR00744)
h. BD Cytofix fixation buffer (BD 554655), BD Phosflow Perm Buffer IV 10× (BD 560746)
i. Antibodies: PerCP/CY5.5-anti-CD3 (Biolegend 317446) APC-Cy7-anti-CD8 (Biolegend 344714),
j. FITC-anti-CD4 (Biolegend 300538), PE-anti-p-Stat4 (BD 562073),
k. BV605-anti-CD56 (Biolegend, 362538), BV421-anti-Lag3 (BD 565720)
l. Human Interleukin-12 (Prospec, #CYT-101 b

The procedure was in the pSTAT4 assay to test the ex vivo biological activities of the IL-12 chimeric molecules is described below:

Healthy human PBMC were isolated from human buffy coat with Ficoll-paque PLUS. Fresh Isolated or frozen PBMCs (60 million) were cultured with 10% FBS in RMPI 1640 complete medium in anti-CD3 (2.5 μg/ml) pre-coated 6-well plate with anti-CD28 (5 μg/ml) and IL-2 (5 ng/ml) for 48 hours.

Activated PBMCs were washed with RPMI 1640 medium and then resuspended in T cell culture medium for overnight culture. Rested PBMC were collected and stained with PerCP/CY5.5 anti-CD3, APC/Cy7-anti-CD8, FITC-anti-CD4, and BV605-anti-CD56 on ice for one-half hour. Anti-Lag3-IL-12 mutein (Lag3-129C) and control reference were serial diluted in 96-well plates and mixed with PBMCs (6×105/well), followed by incubation at 37° c. incubator for 15 minutes. Following cell treatment, an equal volume of warmed BD Cytofix fixation buffer (BD554655) was added to stop the reaction. Cells were washed with PBS, and permeabilized with Perm Buffer IV (0.5×) for 20 minutes. After washing, cells were resuspended in stain buffer with PE-anti-p-Stat4 and BV421-anti-Lag3. Cells were measured by BD flow cytometry (FACSCelesta, BD Biosciences).

### Example 9. Screening of p40 Subunit Mutations and p35 Subunit Mutations

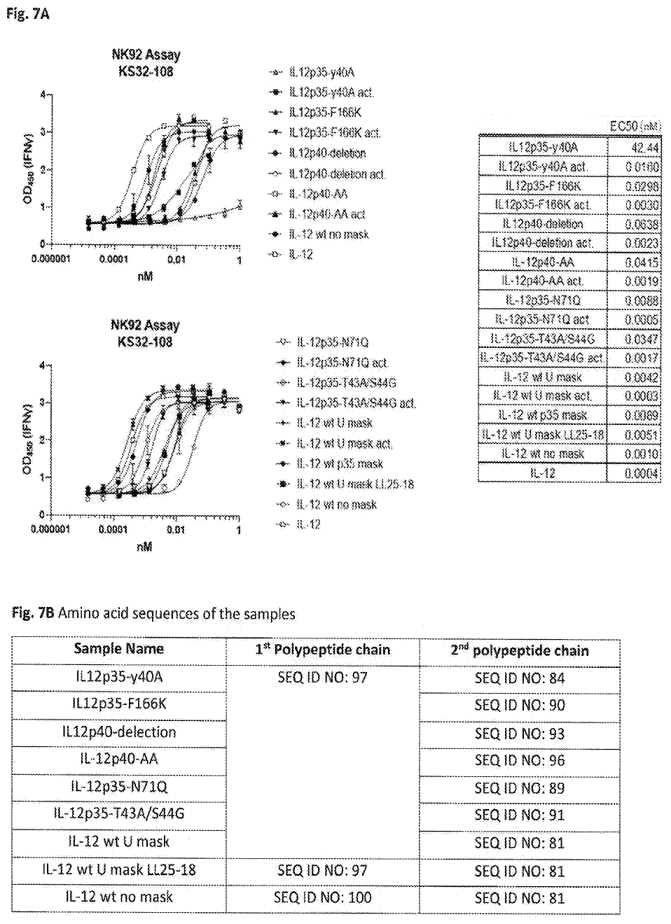

Chimeric molecules which comprise mutant p40 subunit or mutant p35 subunit were expressed and their activities tested. The chimeric molecules comprise mutations and amino acid sequences as shown in FIG. 7B. A control molecule which comprises wild type IL-12 and another control molecule which does not comprises any masking moiety are also included. FIG. 7A shows the NK92 activity assay (Interferon gamma releasing) results.

The results showed that the Y40A mutation in the p35 subunit significantly reduced the activity of the chimeric molecule both prior to activation (an EC50 of 42 nM compared to the EC50 of 0.0042 nM for the wild type IL-12, a 10,000 fold reduction) and after activation (0.0160 nM vs 0.0003 nM, a 50 folder reduction). It was surprising that the change in activity prior to activation was much higher than that after activation, indicating the synergistic interaction between the masking effect and effect from the Y40A mutation. Several additional mutations also induced significant reduction in the activities of the chimeric molecules prior to and after activation, which include: (a) p35-F166K, a 7 folder reduction prior to activation, and a 10 folder reduction after activation; (b) p40-deletion, a 15 folder reduction prior to activation, and about an 8 folder reduction after activation; (c) p40-AA (K260A/R261A), a 10 folder reduction prior to activation, and a five folder reduction after activation; and (d) p35-T43A/S44G, an 8 folder reduction prior to activation, and a four folder reduction after activation. All those mutations are of potential applications when a lower potency or safer molecule is needed. Alternatively, those mutations may provide selectivity of the IL-12 chimeric molecules with certain subsets or subsets of immune cells.

Ex vivo biological activities of the chimeric molecules were also analyzed using Interferon-y releasing assay (FIGS. 10A and 10B). The results showed that both masking moieties significantly reduced the activity of IL-12 in this Interferon-γ releasing assay. In addition, p35-Y40A mutation further reduced the activities of the chimeric molecule with U mask.

### Example 10. Linker Screening for Chimeric Molecules Comprising Single Domain Antibodies which Bind to LAG-3

IL-12 chimeric molecules are complex molecules and aggregation formation was one of the main challenges for their productions. In order to design better molecules, the linkers in the chimeric molecules were tested. Chimeric molecules comprising single domain antibodies against LAG-3 were expressed and purified by Protein A affinity chromatography. The sequence information of the chimeric molecules is shown in Table 6. All the molecules comprise a general structure as illustrated in FIG. 8. The molecules have various length of the peptide linker linking the masking moiety to the C-terminus of one of the Fc polypeptides, GGGGSGGGGSAAGGGGSGGGGS, GGGGSGGGGSGGGGS, GGGGSGGGGS, and GGGGS. The peptide linker linking the p40 subunit to the other Fc polypeptide comprises GGGGSGGGGSGGGGS. And the peptide linker linking the p35 subunit to the p40 subunit also comprises GGGGSGGGGSGGGGS. In general, the SEC-HPLC main peak purities were low (<35%) (Table 6).

TABLE 6
SEC-HPLC Purity and Sequence Information of Chimeric Molecules with
Various Peptide Linkers Which Link The Masking Moiety to The Carrier

| ExpiCHO | | | | | |
|---|---|---|---|---|---|
| Expression | | | | Linker between | |
| Sample | | | | Masking Moiety | SEC-HPLC, |
| Name | Fc-cytokine | Fc-Mask | ratio | and Carrier | Main Peak % |
| JR11.159.1 | CX14.51.3 | CX14.51.1 | 1:1 | 2xG4SAA2xG4S | 34.93 |
| | SEQ ID NO: 124 | (SEQ ID NO: 115) | | | |
| JR11.159.2 | | CX14.72.1 | 1:1 | 3xG4S | 25.23 |
| | | (SEQ ID NO: 111) | | | |
| JR11.159.3 | | CX14.72.2 | 1:1 | 2xG4S | 28.04 |
| | | (SEQ ID NO: 112) | | | |
| JR11.159.4 | | CX14.72.3 | 1:1 | 1xG4S | 26.79 |
| | | (SEQ ID NO: 113) | | | |

In another attempt, the peptide linker linking the p35 subunit to the p40 subunit was shorter, comprising GGGGS, or GGGGSGGGGS. Surprisingly, the SEC-HPLC main peak purities were significantly higher, ranged from 47% to 54% (Table 7). To their surprise, the inventors of this invention discovered that the product purity can be improved significantly by reducing the length of the linker between the p40 subunit and the p35 subunit in the chimeric molecule.

TABLE 7
SEC-HPLC Purity and Sequence Information of Chimeric Molecules
with Shorter Peptide Linkers between p40 and p35

| | | | | Linker | | |
|---|---|---|---|---|---|---|
| | | | | Linking the | Linker | |
| Sample | Cytokine | Mask | | Mask to the | Connecting | SEC-HPLC |
| Name | Chain | Chain | ratio | Carrier | p35-p40 | Main Peak % |
| SR02.2.1 | CX14.74.1 | CX14.51.1 | 1:1 | 2xG4SAA2xG4S | 2xG4S | 51.48% |
| | SEQ ID | SEQ ID | | | | |
| | NO: 116 | NO: 115 | | | | |
| SR02.2.2 | CX14.74.2 | | 1:1 | | 1xG4S | 52.16% |
| | SEQ ID | | | | | |
| | NO: 117 | | | | | |
| SR02.2.3 | CX14.74.1 | CX14.72.4 | 1:1 | No linker | 2xG4S | 47.00% |
| | SEQ ID | SEQ ID | | | | |
| | NO: 116 | NO: 114 | | | | |
| SR02.2.4 | CX14.74.2 | | 1:1 | | 1xG4S | 51.23% |
| | SEQ ID | | | | | |
| | NO: 117 | | | | | |
| SR02.2.5 | CX14.74.1 | | 2:1 | | 2xG4S | 53.96% |
| | SEQ ID | | | | | |
| | NO: 116 | | | | | |

### Example 12. Bioassay Activity Characterization of Chimeric Molecules Comprising a Single Domain Antibody Against LAG-3

Several chimeric molecules comprising a single domain antibody against LAG-3, ASKG129A, ASKG129B and ASKG129C were expressed and purified (Table 5). The biological activities of those chimeric molecules were analyzed.

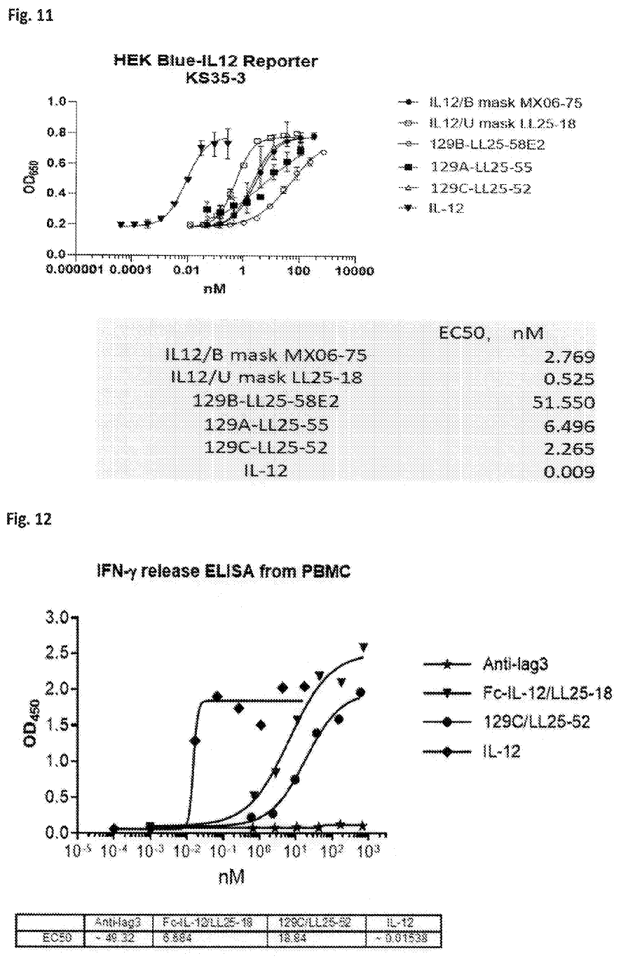

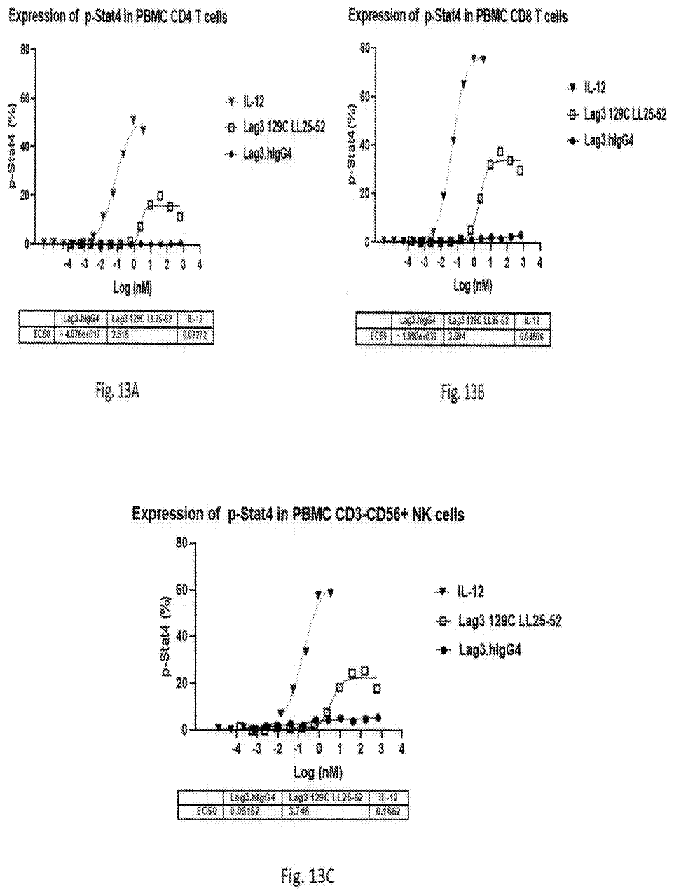

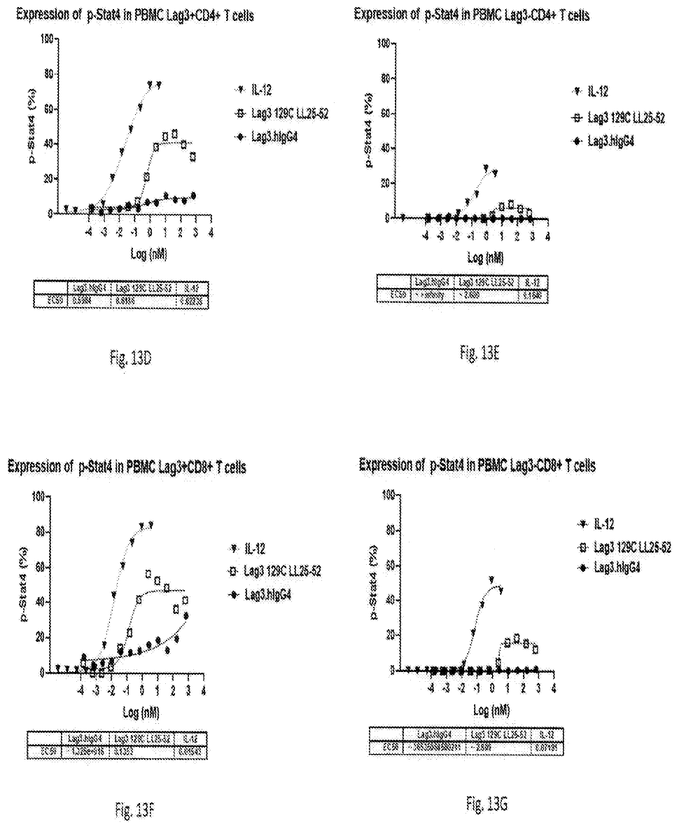

FIG. 11 shows the results of the HEK Blue-IL12 assay for the purified chimeric molecules ASKG129A, ASKG129B and ASKG129C. The data showed that all three molecules had significantly reduced biological activities as compared with IL-12. EC50 values of the molecules were over 200 times to 5000 times larger than IL-12. Notably, the masking moiety and the p35-Y40A mutation together significantly reduced the activities of IL-12. FIG. 12 shows the cytokine releasing assay with human PBMC. The activity of ASKG129C was reduced by approximately 1000 times as that of IL-12, which may make this chimeric molecule significantly safer for therapeutic applications.

FIGS. 13A-13F show the results of the ex vivo pSTAT4 analysis. Human recombinant IL-12 induced dose-dependent phosphorylation of STAT4 (p-STAT4) in human PBMC (EC50 values CD4⁺ T cells: 0.072 nM, CD8⁺ T cells: 0.045 nM, NK cells: 0.1662 nM), compared with Lag3-129C (or ASKG129C) (EC50 values CD4⁺ T cells: 2.52 nM, CD8⁺ T cells: 2.09 nM, NK cells: 3.75 nM). The E-max (Maximal response) for human IL-12-induced CD8⁺ T cells and NK cells was around 60 to 80% p-STAT4 positive cells vs 40 to 50% p-STAT4 positive CD4⁺ T cells. However, the Lag3-129C (or ASKG129C)-induced Emax response was much reduced in CD8⁺ T cells, NK cells, and CD4⁺ T cells (30%, 20%, and 10%) respectively. Consistent with the other two in vitro or ex vivo assays, the activity of Lag3-129C to induce activation of STAT4 was significantly reduced compared with human recombinant IL-12 in CD4⁺ T cells, CD8⁺ T cells and NK cells.

Surprisingly, further analysis showed that ASKG129C induced phosphorylation of STAT4 in the majority of Lag3⁺CD4⁺ and Lag3⁺CD8⁺ T cells and demonstrated significantly higher biological activities compared with the Lag3-CD4⁺ and Lag3-CD8W T cells. The inventor of this invention made a surprising discovery that the masked IL-12 polypeptide was able to activate cells expressing the antigen (in this case LAG3) targeted by the antigen-binding moiety in the carrier even though the masking moiety of the chimeric molecule was not cleaved or removed.

As a control, anti-Lag3 hIgG4 show no detectable ability to induce phosphorylation of STAT4 in the majority of immune cells (CD4⁺ T, CD8⁺ T cells, and NK cells).

### Example 13. Expression and Characterization of Chimeric Molecules Comprising An Antibody Against PD-1

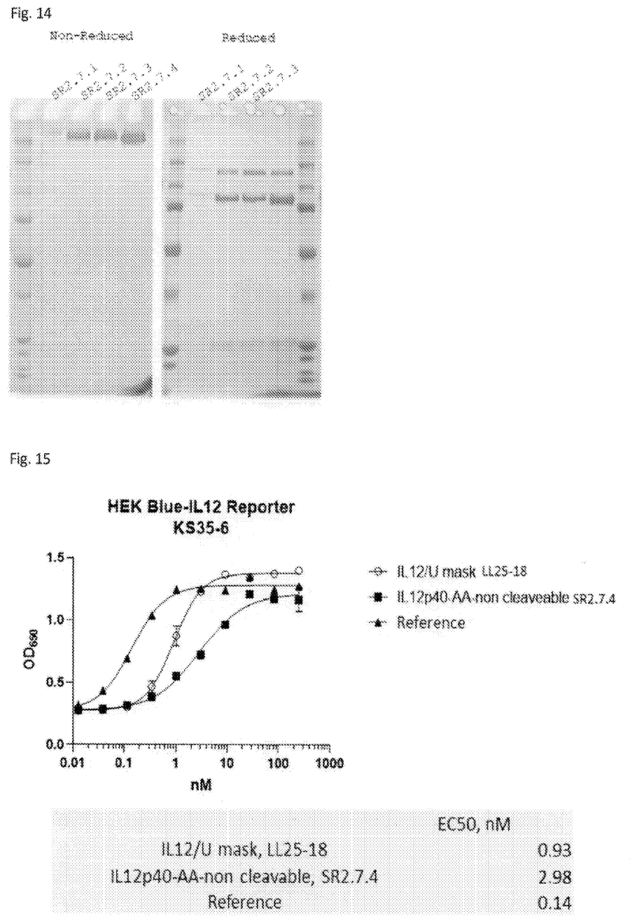

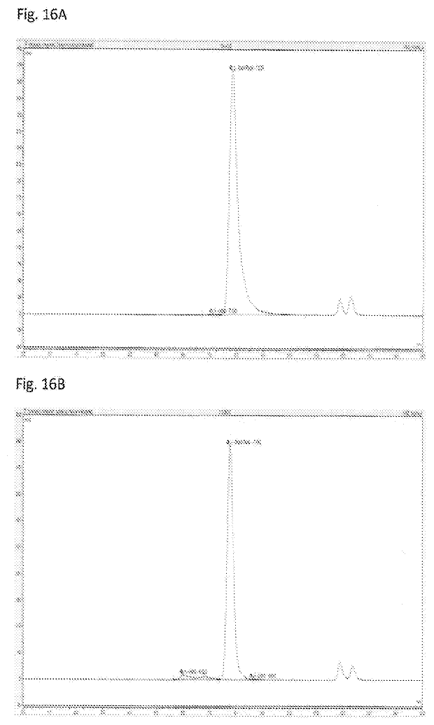

Chimeric molecules comprising an anti-PD-1 antibody as their carrier were also expressed and purified. Table 8 shows the amino acid sequence information of the chimeric molecules. FIG. 14 shows the purity analysis of the chimeric molecules by reduced and non-reduced SDS-PAGE. FIG. 15 shows the HEK-Blue IL-12 Assay results for the chimeric molecule which comprises an anti-PD-1 antibody and an IL-12 mutant, wherein a mutant comprises p40-K260A/R261A mutations. The activity of a chimeric molecule was lower compared to the Fc-based IL-12 chimeric molecule which comprises the wild type IL-12 and is masked by the U mask. In addition, both molecules had lower activity compared to the reference molecule, which is an IL-12 mutant molecule fused to the Fc domain, which was disclosed in United States Patent Application 20200216509. The results showed that chimeric molecule comprising an anti-PD-1 antibody as the carrier can be expressed and purified, wherein a chimeric molecule further showed expected biological activity based on the reporter assay.

TABLE 8
Sequence Information of Chimeric Molecules Comprising
An Anti-PD-1 Antibody As the Carrier

| | | | | | | | Plasmid |
|---|---|---|---|---|---|---|---|
| Molecule | Chain 1 | | Chain 2 | | Chain 3 | | ratio |
| SR2.7.1 | CX14.77.1 | SEQ ID | CX14.77.4 | SEQ ID | CX11.135.1 | SEQ ID | 2:1:3 |
| SR2.7.2 | | NO: 125 | | NO: 128 | | NO: 129 | 1:1:2 |
| SR2.7.3 | CX14.77.3 | SEQ ID | | | | | 2:1:3 |
| SR2.7.4 | | NO: 127 | | | | | 1:1:2 |

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

It is understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is not intended to be limited to a particular compound, composition, article, or method, unless expressly stated as such. In addition, those of ordinary skill in the art will recognize that certain changes, modification, permutations, alterations, additions, subtractions, and sub-combinations thereof can be made in accordance with the teachings herein without departing from the spirit of the present specification.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

1. A chimeric molecule, which comprises an IL-12 p40 subunit (p40), an IL-12 p35 subunit (p35), a masking moiety (MM), and a carrier (C); wherein:
(a) said IL-12 subunit p40 comprises an amino acid sequence of SEQ ID NO: 5 or an amino acid sequence with at least 95% identity to that of SEQ ID NO: 5;
(b) said IL-12 subunit p35 comprises an amino acid sequence of SEQ ID NO: 6 or an amino acid sequence with at least 95% identity to that of SEQ ID NO: 6;
(c) said masking moiety comprises an amino acid sequence of SEQ ID NOS: 1, 2, 3 or 4 or an amino acid sequence with at least 95% identity to that of SEQ ID NO: 1, 2, 3 or 4;
(d) said carrier is selected from an albumin, an albumin fragment, an Fc domain, and an antibody; and
(e) said IL-12 p40 subunit comprises one or more mutations at one or more sites selected from the following: Lys258, Ser259, Lys260, Arg261, Lys263, Lys264, Asp265, and Arg266 (numbering according to SEQ ID NO: 5).

2. A chimeric molecule, which comprises an IL-12 p40 subunit (p40), an IL-12 p35 subunit (p35), a masking moiety (MM), and a carrier (C); wherein:
(a) said IL-12 subunit p40 comprises an amino acid sequence of SEQ ID NO: 5, or an amino acid sequence with at least 95% identity to that of SEQ ID NO: 5;
(b) said IL-12 subunit p35 comprises an amino acid sequence of SEQ ID NO: 6, or an amino acid sequence with at least 95% identity to that of SEQ ID NO: 6;
(c) said masking moiety comprises an amino acid sequence of SEQ ID NO: 1, 2, 3 or 4, or an amino acid sequence with at least 95% identity to that of SEQ ID NO: 1, 2, 3 or 4;
(d) said carrier is selected from an albumin, an albumin fragment, an Fc domain, and an antibody; and
(e) said IL-12 p40 subunit comprises mutations Lys260Ala and Arg261Ala.

3. A chimeric molecule, which comprises an IL-12 p40 subunit (p40), an IL-12 p35 subunit (p35), a masking moiety (MM), and a carrier (C); wherein:
(a) said IL-12 subunit p40 comprises an amino acid sequence of SEQ ID NO: 46, 63, or 98, or an amino acid sequence with at least 99% identity to that of SEQ ID NO: 46, 63, or 98;
(b) said IL-12 subunit p35 comprises an amino acid sequence of SEQ ID NO: 6 or an amino acid sequence with at least 95% identity to that of SEQ ID NO: 6;
(c) said masking moiety comprises an amino acid sequence of SEQ ID NO: 1, 2, 3 or 4 or an amino acid sequence with at least 95% identity to that of SEQ ID NO: 1, 2, 3 or 4; and
(d) said carrier is selected from an albumin or albumin fragment, an Fc domain, and an antibody.

4. The chimeric molecule of claim 1, wherein:
(a) said masking moiety is linked to the carrier through a cleavable peptide linker, or a non-peptide cleavable linker;
(b) said p40 subunit is fused to the carrier through a non-cleavable linker; and
said p35 subunit is fused to the p40 subunit through a non-cleavable linker.

5.-18. (canceled)

19. The chimeric molecule of claim 1, wherein the carrier comprises an antigen binding moiety, which binds to an antigen expressed on the surfaced of a tumor cell, a cancer cell, or an immune cell; wherein said antigen is selected from PD-1, PD-L1, CD47, CD16a, NKG2D, NKG2A, SIRPα, Siglec-10, TIGIT, TIM-3, LAG-3, 5T4, FAP, EGFR, CMET, HER2, Trop-2, VEGFR2, ROR1, mesothelin, GPC-3, CD20, CD38, and BCMA.

20.-27. (canceled)

28. A chimeric molecule, which comprises an IL-12 p40 subunit (p40), an IL-12 p35 subunit (p35), a masking moiety (MM), and a carrier (C); wherein:
(a) said IL-12 subunit p40 comprising an amino acid sequence of SEQ ID NO: 5 or an amino acid sequence of at least 95% identity to that of SEQ ID NO: 5;
(b) said IL-12 subunit p35 comprising an amino acid sequence of SEQ ID NO: 6 or an amino acid sequence of at least 95% identity to that of SEQ ID NO: 6;
(c) said masking moiety comprises an amino acid sequence of SEQ ID NOS: 1, 2, 3 or 4, or an amino acid sequence of at least 95% identity to that of SEQ ID NO: 1, 2, 3 or 4;
(d) said carrier comprises an antigen-binding moiety which binds to PD-1 and an Fc domain with a first Fc polypeptide and a second Fc polypeptide;
(e) said masking moiety if fused to the C-terminus of the first Fc polypeptide with or without a non-cleavable or cleavable peptide linker; wherein said peptide linker optionally comprises an amino acid sequence of 3 amino acids to 22 amino acids in length;
(f) said p40 subunit is fused to the 2^{nd} Fc polypeptide optionally through a non-cleavable peptide linker;
(g) said p35 subunit is fused to the C-terminus of the p40 subunit optionally through a peptide linker
wherein said chimeric molecule has higher activity stimulating a PD-1 positive (or PD-1⁺) T cell compared to that of stimulating an NK cell.

29.-31. (canceled)

32. The chimeric molecule of claim 28 which comprises two polypeptide chains; wherein said first polypeptide chain comprises an amino acid sequence selected from SEQ ID NOs: 103-108 or an amino acid sequence of at least 99% identity to one of SEQ ID NOs: 103-108; and wherein said second polypeptide chain comprises an amino acid sequence selected from SEQ ID NOs: 109 and 110 or an amino acid sequence of at least 99% identity to one of SEQ ID NOs: 109 or 110.

33. The chimeric molecule of any of claim 28 which comprises two identical light chains and a first heavy chain polypeptide chain and a second heavy chain polypeptide chain; wherein said light chains comprise an amino acid sequence of SEQ ID NO: 129 or an amino acid sequence of at least 95% identity to SEQ ID NO: 129; wherein said first heavy chain polypeptide chain comprises an amino acid sequence selected from SEQ ID NOs: 125-127 or an amino acid sequence of at least 99% identity to SEQ ID NOs: 125-127; and wherein said second heavy chain polypeptide chain comprises an amino acid sequence as SEQ ID NO: 128 or an amino acid sequence of at least 99% identity to SEQ ID NO: 128.

34. The chimeric molecule of any of claim 28 which comprises two identical light chains and a first heavy chain polypeptide chain, and a second heavy chain polypeptide chain; wherein said light chains comprise an amino acid sequence of SEQ ID NO: 129 or an amino acid sequence of at least 95% identity to SEQ ID NO: 129; wherein said first heavy chain polypeptide chain comprises an amino acid sequence selected from SEQ ID NOs: 130-135 or an amino acid sequence of at least 99% identity to one of SEQ ID NOs: 130-135; and further wherein said second heavy chain polypeptide chain comprises an amino acid sequence of SEQ ID NO: 136 or an amino acid sequence of at least 99% identity to one of SEQ ID NO: 136.

35.-37. (canceled)

38. A pharmaceutical composition comprising the chimeric molecule of claim 28 as its active ingredient and a pharmaceutically acceptable excipient.

39.-47. (canceled)

48. The chimeric molecule of claim 2, wherein:
(a) said masking moiety is linked to the carrier through a cleavable peptide linker, or a non-peptide cleavable linker;
(b) said p40 subunit is fused to the carrier through a non-cleavable linker; and
said p35 subunit is fused to the p40 subunit through a non-cleavable linker.

49. The chimeric molecule of claim 3, wherein:
(a) said masking moiety is linked to the carrier through a cleavable peptide linker, or a non-peptide cleavable linker;
(b) said p40 subunit is fused to the carrier through a non-cleavable linker; and
said p35 subunit is fused to the p40 subunit through a non-cleavable linker.

50. The chimeric molecule of claim 2, wherein the carrier comprises an antigen binding moiety, which binds to an antigen expressed on the surfaced of a tumor cell, a cancer cell, or an immune cell; wherein said antigen is selected from PD-1, PD-L1, CD47, CD16a, NKG2D, NKG2A, SIRPα, Siglec-10, TIGIT, TIM-3, LAG-3, 5T4, FAP, EGFR, CMET, HER2, Trop-2, VEGFR2, ROR1, mesothelin, GPC-3, CD20, CD38, and BCMA.

51. The chimeric molecule of claim 3, wherein the carrier comprises an antigen binding moiety, which binds to an antigen expressed on the surfaced of a tumor cell, a cancer cell, or an immune cell; wherein said antigen is selected from PD-1, PD-L1, CD47, CD16a, NKG2D, NKG2A, SIRPα, Siglec-10, TIGIT, TIM-3, LAG-3, 5T4, FAP, EGFR, CMET, HER2, Trop-2, VEGFR2, ROR1, mesothelin, GPC-3, CD20, CD38, and BCMA.
